(19) United States
(12) Patent Application Publication
(10) Pub. No.: US 2026/0286352 A1
(43) Pub. Date: Sep. 24, 2026

(54) PROGRAMMABLE RNA SCAFFOLDS FOR MULTIVARIATE EFFECTOR RECRUITMENT USING CRISPR/CAS

(71) Applicant: William Marsh Rice University, Houston, TX (US)

(72) Inventors: Isaac HILTON, Houston, TX (US); Barun MAHATA, Houston, TX (US); Alan CABRERA, Houston, TX (US)

(73) Assignee: William Marsh Rice University, Houston, TX (US)

(21) Appl. No.: 19/484,571

(22) PCT Filed: May 17, 2024
(86) PCT No.: PCT/US2024/029912

§ 371 Date: Nov. 14, 2025

Related U.S. Application Data
(60) Provisional application No. 63/467,126, filed on May 17, 2023.

(51) Int. Cl. C12N 15/11; C12N 9/22; C12N 15/86

(52) U.S. Cl. CPC C12N 15/111; C12N 9/226; C12N 15/86; C07K 2319/71; C07K 2319/80; C12N 2310/20; C12N 2740/15043; C12N 2830/003

(57) ABSTRACT

The present disclosure is directed to CRISPR systems comprising an RNA scaffold that can recruit up to four different effector domains to human loci. The RNA scaffolds may comprise at least three different types of stem loops that each specifically bind to a different stem-loop binding protein. The stem-loop binding proteins may then be fused to the different effector domains, one of which is a Cas9 protein, to bring all of the components together at one site.

### REFERENCE TO RELATED APPLICATIONS

The present application claims the priority benefit of U.S. provisional application No. 63/467,126, filed May 17, 2023, the entire contents of which are incorporated herein by reference.

### STATEMENT OF FEDERALLY SPONSORED RESEARCH

This invention was made with government support under Grant Nos. R35GM143532 and R56HG012206 awarded by the National Institutes of Health. The government has certain rights in the invention.

### REFERENCE TO A SEQUENCE LISTING

This application contains a Sequence Listing XML, which has been submitted electronically and is hereby incorporated by reference in its entirety. Said Sequence Listing XML, created on May 16, 2024, is named RICEP0135WO_ST26.xml and is 13,237 bytes in size.

### BACKGROUND

### 1. Field

The present disclosure relates generally to the fields of molecular biology, gene regulation and gene therapy. More particular, the disclosure relates to an RNA intermediate effector recruitment platform that permits robust stochiometric and spatiotemporal control over effector recruitment in CRISPR/Cas systems.

### 2. Description of Related Art

Rational engineering of RNA has enabled diverse applications ranging from COVID vaccines to improved guide RNA (gRNAs) for CRISPR/Cas systems in vivo and in vitro. For instance, nearly all CRISPR/Cas9-based genome editing applications rely upon gRNAs that are synthetic fusions between the CRISPR and trans-activating CRISPR RNAs (crRNAs and tracrRNAs, respectively). Further, chemical modifications to gRNAs are often required for genome editing in cell and gene therapy contexts as well as CRISPR activation (CRISPRa) strategies. gRNAs are frequently modified to incorporate stem-loops (SLs) that can recruit stem-loop binding proteins (SLBPs) fused to different transcriptional effectors. However, these stem-loop platforms currently offer limited spatiotemporal control and can sometimes even interfere with gRNA efficacy. Improved methods of employing CRISPR/Cas systems that are not so limited are of great interest and need.

### SUMMARY

Thus, provided herein are compositions comprising one or more nucleic acid(s) encoding (i) a first effector protein fused to a first stem-loop binding protein, wherein the first effector protein is a CRISPR/Cas protein, (ii) a second effector protein fused with a second stem-loop binding protein, (iii) an RNA molecule (a.k.a., designer RNA (dRNA) or bridge RNA) comprising at least one of a first stem loop that binds to the first stem-loop binding protein and at least one of a second stem loop that binds to the second stem-loop binding protein, and (iv) one or more guide RNA (gRNA) targeting one or more genomic locus. The components may all be present on a single nucleic acid molecule. Also provided herein are cells comprising (i) a first effector protein fused to a first stem-loop binding protein, wherein the first effector protein is a CRISPR/Cas protein, (ii) a second effector protein fused with a second stem-loop binding protein, (iii) an RNA molecule comprising at least one of a first stem loop that binds to the first stem-loop binding protein and at least one of a second stem loop that binds to the second stem-loop binding protein, and (iv) one or more guide RNA (gRNA) targeting one or more genomic locus. Also provided herein are viral vectors (e.g., lentiviral vectors or adenovirus associated vectors) comprising nucleic acid sequences encoding (ii) a second effector protein fused with a second stem-loop binding protein, (iii) an RNA molecule comprising at least one of a first stem loop that binds to the first stem-loop binding protein and at least one of a second stem loop that binds to the second stem-loop binding protein, and (iv) one or more guide RNA (gRNA) targeting one or more genomic locus. The gRNA and the dRNA molecule may each be under the control of a polIII promoter, e.g., an RNA polymerase III 7SK promoter. The stem loop:stem-loop binding protein pairs may comprise PP7:PCP, MS2:MCP, CsrB-SL:CsrA, RAT:LicT, and/or RAT:LicV.

The CRISPR/Cas may be dCas9, SadCas9, CjdCas9, SpdCas9, SpRY dCas9, or HIFI dCas9. The CRISPR/Cas may be fused to two or more copies of the first stem-loop binding protein. The CRISPR/Cas may be fused to at least 1, 2, 3, or 4 copies of the first stem-loop binding protein. The CRISPR/Cas may be N-terminally and/or C-terminally fused to the at least 1, 2, 3, or 4 copies of the first stem-loop binding protein. The CRISPR/Cas may be N-terminally fused to 1 copy of the first stem-loop binding protein. The CRISPR/Cas may be C-terminally fused to 1 copy of the first stem-loop binding protein. The CRISPR/Cas may be N-terminally fused to 1 copy of the first stem-loop binding protein and C-terminally fused to 1 copy of the first stem-loop binding protein. The CRISPR/Cas may be N-terminally fused to 2 copies of the first stem-loop binding protein. The CRISPR/Cas may be C-terminally fused to 2 copies of the first stem-loop binding protein. The CRISPR/Cas may be N-terminally fused to 2 copies of the first stem-loop binding protein and C-terminally fused to 2 copies of the first stem-loop binding protein. The CRISPR/Cas may be N-terminally fused to 3 copies of the first stem-loop binding protein. The CRISPR/Cas may be C-terminally fused to 3 copies of the first stem-loop binding protein. The CRISPR/Cas may be N-terminally fused to 3 copies of the first stem-loop binding protein and C-terminally fused to 3 copies of the first stem-loop binding protein. The CRISPR/Cas may be N-terminally fused to 4 copies of the first stem-loop binding protein. The CRISPR/Cas may be C-terminally fused to 4 copies of the first stem-loop binding protein. The CRISPR/Cas may be N-terminally fused to 4 copies of the first stem-loop binding protein and C-terminally fused to 4 copies of the first stem-loop binding protein.

The dRNA molecule comprises two, three, four or more copies of the first stem loop; and two, three, four or more copies of the second stem loop. The dRNA molecule may comprise two copies of the first stem loop and two, three, four or more copies of the second stem loop. The dRNA molecule may comprise two copies of the second stem loop and two, three, four or more copies of the first stem loop. The dRNA molecule may comprise two copies of the first stem loop in tandem with two copies of the second stem loop. The dRNA molecule may comprise two copies of the first stem loop alternating with two copies of the second stem loop. The dRNA molecule may comprise four copies of the first stem loop in tandem with four copies of the second stem loop. The dRNA molecule may comprise four copies of the first stem loop alternating with four copies of the second stem loop.

There may be a third effector protein fused with a third stem-loop binding protein, in which case the dRNA molecule may further comprise at least one of a third stem loop that binds to the third stem-loop binding protein. When there is a third stem loop, the dRNA molecule comprises two, three, four or more of the third stem loop. There may be a fourth effector protein fused with a fourth stem-loop binding protein, in which case the dRNA molecule may further comprise at least one of a fourth stem loop that binds to the fourth stem-loop binding protein. When there is a fourth stem loop, the dRNA molecule comprises two, three, four or more of the fourth stem loop. The dRNA molecule may comprise 2-4 PP7 stem loops, 2-4 MS2 stem loop, and 2-4 RAT stem loops, and wherein one effector protein is fused to PCP, one effector protein is fused to MCP, and one effector protein is fused to LicT or LicV. The RNA molecule may further comprise at least two CsrB-SL stem loops, wherein one effector protein is fused to CsrA. Each of the effector proteins may be, independently, a transactivating domain or epigenetic editor. Each of the effector proteins may be, independently, selected from VP64, eN3×9, TET1, and MSN.

The dRNA molecule may further comprise a small-molecule cleavable or self-cleavable riboswitch. The dRNA molecule may further comprise a self-stabilized and small molecule/biomolecule induced cleavable OFF-Riboswitch. The riboswitch may be positioned between the tandem copies of the first stem loop and the second stem loop. The riboswitch may be cleaved when bound by guanine. The riboswitch may be a GuaM8HDV riboswitch.

The dRNA molecule may further comprise a small-molecule stabilized or self-stabilized riboswitch. The dRNA molecule may further comprise a small molecule/biomolecule stabilized ON-Riboswitch. The riboswitch may be positioned between the tandem copies of the first stem loop and the second stem loop. The riboswitch may be stabilized when bound by tetracycline. The riboswitch may be a K19 riboswitch.

The dRNA molecule may comprise a stem loop that only binds to its stem-loop binding protein in the presence of blue light. The interaction between the first stem loop and the first stem-loop binding protein may be light dependent. The interaction between the second stem loop and the second stem-loop binding protein may be light dependent. The interaction between the third stem loop and the third stem-loop binding protein may be light dependent.

Also provided herein are methods of modifying a phenotype of a cell comprising introducing a composition or vector described herein into the cell. The phenotype may be an expression level of one or more genes and/or an epigenetic landscape. The one or more genes may encode any of mRNA, lncRNA, and eRNA.

Also provided herein are methods of modulating cholesterol to testosterone production (i.e., stimulating testosterone production) in a cell comprising introducing a composition or vector described herein into the cell. The method may activate the expression of STAR, CYP11A1, HSD3B2, CYP17A1, and HSD17B1. The composition may comprise a gRNA array consisting of gRNAs targeting STAR, CYP11A1, HSD3B2, CYP17A1, and HSD17B1.

Also provided herein are engineered stem loops that specifically bind to CsrA. The engineered stem loop may be CsrB-SL Type 1 or CsrB-SL Type 2. The engineered stem loop may comprise or consist of the RNA sequence of SEQ ID NO: 9. The engineered stem loop may comprise or consist of the RNA sequence of SEQ ID NO: 10. The engineered stem loop may be in a composition that further comprises CsrA (SEQ ID NO: 11).

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D. Identification and selection of optimal DRIMER platform components. FIG. 1A. Schematics showing different dCas9 fusions to RNA binding protein PCP in 1×, 2×, 3× and 4× manner along with N-terminal, C-terminal or both N and C terminal fusion. FIG. 1B. Different designer multi stem-loop structured non-coding RNA are shown. All dRNA contains MS2 and PP7 stem loops. RNA structures were predicted using RNAfold (http://rna.tbi.univie.ac.at/cgi-bin/RNAWebSuite/RNAfold.cgi). FIG. 1C. OCT4 mRNA levels after the indicated DRIMER platforms were recruited to OCT4 promoter via gRNAs. MCP fused to the bipartite p65-HSF was used as transcriptional activator. OCT4 mRNA expression was analyzed by QPCR and presented as fold change after normalization dCas9 control. FIG. 1D. EGFP protein expression after the indicated DRIMER platforms were recruited to mini-CMV promoter driving EGFP expression one gRNA. MCP fused to the tripartite MSN was used as transcriptional activator. EGFP protein expression was analyzed by flow cytometry and presented as MFI fold change after normalization to dCas9 control.

FIGS. 2A-2M. DRIMER mediated programmable gene activation in human cells. FIG. 2A. Schematic showing components of the DRIMER system i) gRNA loaded CRISPR/Cas ribonucleoprotein. ii) Designer RNA (dRNA) with modular stem loop (SL) structures. iii) Stem loop binding protein (SLBP) fused effector. FIGS. 2B and 2C. Relative expression of IL1RN and SBNO2 mRNA levels following transfection of DRIMER system components including 2×PCP-dCas9, 4×PP7-4×MS2 dRNA, MCP fused to tripartite transcriptional activator MSN and 4 gRNA pool for IL1RN or single gRNA for SBNO2. FIG. 2D. Schematic representation showing different MCP fused transcriptional activator proteins used using DRIMER based recruitment. FIG. 2E. Relative expression of IL1RN mRNA level following transfection with indicated MCP-fused effector proteins along with other DRIMER components. FIG. 2F. Relative expression of different coding and non-coding RNA levels following transfection with the canonical DRIMER (2×PCP-dCas9, 4×PP7-4×MS2 dRNA, and MCP-MSN) and respective gRNA pools. FIG. 2G. Relative expression of IL1RN and SBNO2 mRNA levels following transfection of DRIMER delivering dRNA and gRNA from a single construct. FIG. 2H. mRNA levels of STAR, CYP11A1, HSD3B2, CYP17A1, and HSD17B1 following transfection of DRIMER with a single gRNA expression plasmid with multiplexed 6 gRNA array targeting these 5 genes responsible for testosterone production in steroidogenesis pathway. FIG. 2I. Secreted testosterone level was measured from cell culture supernatant 72 hrs following transfection of DRIMER with a multiplexed 6 gRNA array gRNA expression plasmid targeting STAR, CYP11A1, HSD3B2, CYP17A1, and HSD17B1. FIGS. 2J and 2K. IL1RN mRNA levels following transfection of engineered PAM flexible or high fidelity engineered SpdCas9 based DRIMER variants in HEK-293T cells respectively. FIGS. 2L and 2M. IL1RN mRNA levels following transfection of Cas9 orthologs including SadCas9 and CjdCas9 based DRIMER variants in HEK-293T cells. All qPCR samples were processed 72 hrs post-transfection and are the result of at least 3 biological replicates.

FIGS. 3A-3D. DRIMER mediated programmable gene activation in diverse human cells. FIGS. 3A and 3B. Relative expression of HBG1 mRNA levels following transfection of DRIMER system components including 2×PCP-dCas9, 4×PP7-4×MS2 dRNA, MCP fused to tripartite transcriptional activator MSN and 4 gRNA pool for HBG1 in HeLa and U20S cells respectively. FIG. 3C. Relative expression of TTN mRNA levels following transfection of DRIMER system components including 2×PCP-dCas9, 4×PP7-4×MS2 dRNA, MCP fused to tripartite transcriptional activator MSN and 4 gRNA pool for TTN in A549 cells. FIG. 3D. Relative expression of MYOD mRNA levels following transfection of DRIMER system components including 2×PCP-dCas9, 4×PP7-4×MS2 dRNA, MCP fused to tripartite transcriptional activator MSN and 4 gRNA pool for MYOD in A549 cells. All qPCR samples were processed 72 hrs post-transfection and are the result of at least 3 biological replicates.

FIGS. 4A-4C. Minimization of DRIMER components to All-in-One (AIO) delivery platform. FIGS. 4A and 4B. Schematic representation showing 2 component and AIO DRIMER platform, respectively. FIG. 4C. Relative expression of TTN mRNA levels following transfection of 2 component and AIO DRIMER platform in HEK293T cells. All qPCR samples were processed 72 hrs post-transfection and are the result of at least 3 biological replicates.

FIGS. 5A-5B. Comparison of DRIMER mediated gene activation with DREAM, Sun-Tag and direct fusion-based gene activation platform. Relative expression of IL1RN and SBNO2 mRNA levels are shown respectively, following transfection of DREAM, DRIMER, Sun-Tag components in HEK293T cells. Tripartite MSN was used in all platforms for transcription activation. All qPCR samples were processed 72 hrs post-transfection and are the result of at least 3 biological replicates.

FIGS. 6A-6G. Small molecule cleavable OFF Riboswitch controlled DRIMER mediated tunable gene expression and metabolic engineering in human cells. FIGS. 6A-6B. Schematic representation showing of OFF Ribo-DRIMER, which utilizes the Guanine responsive cleavable riboswitch GuaMH8HDV containing dRNA (4×PP7-GuaM8HDV-4×MS2). FIG. 6C. Schematic representation showing components of OFF Ribo-DRIMER used for transfection in HEK293Tcells. FIG. 6D. Relative expression of IL1RN mRNA levels following transfection of Ribo-DRIMER with increasing Guanine concentrations 0 μM-800 μM. 400 μM was shown in bold and used for all other experiments FIG. 6E. Schematic of progesterone biosynthetic pathway highlighting key genes and intermediate molecules of the pathway. FIG. 6F. Relative mRNA levels of STAR, CYP17A1, and HSD3B2 following transfection of OFF Ribo-DRIMER with single vector multiplexed 4 gRNA (1 gRNA for STAR, 1 gRNA for CYP17A1, and 2 gRNA for HSD3B2) and Guanine concentrations of either 0 μM or 400 μM. FIG. 6G. Secreted progesterone level was measured from cell culture supernatant 72 hrs following transfection of DRIMER with multiplexed gRNA expression plasmid for STAR, CYP11A1, HSD3B2 in the presence of absence of 400 μM of guanine.

FIGS. 7A-7F. Self-cleavable tetracycline stabilized ON Riboswitch controlled DRIMER mediated tunable gene expression and graded transcriptional response in human cells. FIGS. 7A and 7B. Schematic representation showing of ON Ribo-DRIMER, which utilizes the tetracycline responsive self-cleavable K19 riboswitch containing dRNA (4×PP7-K19-4×MS2). FIG. 7C. Relative expression of IL1RN mRNA (against 0 μM control) levels following transfection of ON Ribo-DRIMER components with increasing tetracycline concentrations 0 μM-100 μM. FIG. 7D. Relative expression of IL1RN mRNA (against nontransfected control) levels following transfection of Ribo-DRIMER with increasing Guanine concentrations 0 μM-800 μM. This data incorporated to demonstrate the leakiness of the system. FIG. 7E. Schematic representation showing destabilization domain DHFR fused MCP-MSN and tetracycline responsive dRNA (4×PP7-K19-4×MS2) used to minimize basal leakiness of the ON Ribo-DRIMER platform. Small molecule TMP (0 nM-1000 nM) was used to stabilize the DHFR-MCP-MSN. FIG. 7F. Relative IL1RN mRNA following transfection of ON Ribo-DRIMER along with DHFR-MCP-MSN followed by increasing concentration of Tetracycline (0 μM-100 μM) and TMP (0 nM-1000 nM).

FIGS. 8A-8D. Light controlled DRIMER mediated gene activation and metabolic engineering. FIG. 8A. Schematic of blue light controllable Opto-DRIMER system. In brief, the dRNA contains 4×PP7 and 4×RAT. 4×PP7 will anchor to dCas9 whereas 4×RAT will bind to LicV (LicT-VVD)-MSN only in the presence of blue light. FIG. 8B. Relative IL1RN mRNA expression level was compared between Opto-DRIMER transfected cells with or without blue light for 24 hrs. FIG. 8C. Schematics showing Cholesterol to Testosterone producing biosynthetic pathway along with respective biosynthetic enzymes. FIG. 8D. Relative mRNA levels of STAR, CYP11A1, HSD3B2, CYP17A1, and HSD17B1 following transfection of Opto-DRIMER with and without blue light in the presence of only dCas9 or 2×PCP-dCas9. All QPCR samples were processed 72 hrs post-transfection and are the result of at least 3 biological replicates.

FIGS. 9A-9C. Doxycycline tunable DRIMER mediated gene activation in human cells. FIG. 9A. Schematic representation of 2 construct delivery strategy utilizing tetracycline inducble 2×PCP-dCas9 and CsrA-eN3×9 and constitutive 4×PP7-12×CsrB-SL dRNA and single gRNA targeting TTN gene promoter. FIG. 9B. Relative expression of TTN mRNA level following transfection of tetracycline inducible DRIMER constructs and addition of doxycline in HEK293T cells. FIG. 9C. Relative expression of TTN mRNA level following transduction of tetracycline inducible DRIMER lentivirus and addition of doxycline in HEK293T cells. All qPCR samples were processed 72 hrs post-transfection and are the result of at least 3 biological replicates.

FIGS. 10A-10J. The novel engineered CsrA: CsrB-SL pair potently induces transcription in combination with DRIMER. FIG. 10A. Schematic representation of 2×PCP-dCas9 and dRNA, where WT CsrB non-coding RNA (SEQ ID NO: 12) was fused to 4×PP7 (4×PP7-CsrB) and human codon optimized WT CsrA or mutant CsrA (H43D) fused to tripartite transcriptional activator MSN. FIG. 10B. Relative IL1RN mRNA expression level following transfection with 4×PP7-CsrB dRNA and either WT CsrA or mutant CsrA H43D fused MSN. FIG. 10C. Schema of engineered type 1 (SEQ ID NO: 9) and type 2 (SEQ ID NO: 10) CsrB SLs and the structure of 4×PP7-4×CsrB-SL dRNA (SEQ ID NO: 13). FIG. 10D. Relative IL1RN mRNA expression level following transfection with indicated CsrA-fused transcriptional activator proteins. FIGS. 10E-10G. Relative IL1RN mRNA expression level following transfection of DRIMER platform containing 2×LicT, 2×MCP, or 2×PCP fused dCas9 and CsrA-MSN along with IL1RN guide pool and 4×RAT-4×CsrA, 4×MS2-4×CsrA, 4×PCP-4×CsrA dRNA respectively. FIGS. 10H-10J. Relative IL1RN mRNA expression level following transfection of DRIMER platform containing 2×CsrA fused dCas9 and 2×LicT, 2×MCP, or 2×PCP fused to MSN along with IL1RN guide pool and 4×RAT-4×CsrA, 4×MS2-4×CsrA, 4×PCP-4×CsrA dRNA respectively. All qPCR samples were processed 72 hrs post-transfection and are the result of at least 3 biological replicates.

FIGS. 11A-11H. The DRIMER system allows combinatorial recruitment identifying synergistic effector relationships in transcriptional regulation. FIG. 11A. Illustration showing schema of the combinatorial recruitment strategy of two transcriptional activators using dRNA. In brief, the designer RNA used here contains 4×PP7-2×MS2-2×RAT SLs, which could recruit two different types of effector proteins fused to MCP (for MS2) and RAT (for LicT) along with PCP fused dCas9. FIG. 11B. Relative IL1RN mRNA expression level following transfection of DRIMER with 4×PP7-2×MS2-2×RAT and MCP-VP64 and LicT-MSN. FIG. 11C. Relative IL1RN mRNA expression level following transfection of DRIMER with 4×PP7-2×MS2-2×RAT and MCP-eN3×9 and LicT-VP64. FIG. 11D. Schema showing combinatorial recruitment strategy of one epigenetic effector (TET1-CD) and one transcriptional activator using dRNA. In brief, the designer RNA used here contains 4×PP7-2×MS2-2×RAT SLs, which could recruit two different types of effector proteins fused to MCP (for MS2) and RAT (for LicT) along with PCP fused dCas9. FIG. 11E. Relative IL1RN mRNA expression level following transfection of DRIMER with 4×PP7-2×MS2-2×RAT and MCP-TET1 and LicT-VP64. FIG. 11F. Relative IL1RN mRNA expression level following transfection of DRIMER with 4×PP7-2×MS2-2×RAT and MCP-TET1 and LicT-eN3×9. FIG. 11G. Illustration showing schema of the combinatorial recruitment strategy of three transcriptional activators using dRNA. In brief, the designer RNA used here contains 2×PP7-2×MS2-2×RAT-2×CsrB-SL, which could recruit three different types of effector proteins fused to MCP (for MS2), RAT (for LicT) and CsrA (2×CsrB-SL) along with 2×PCP fused dCas9. FIG. 11H. Relative IL1RN mRNA expression level following transfection of DRIMER with 2×PP7-2×MS2-2×RAT-2×CsrB: SL containing dRNA and MCP-MRTF-A and LicT-STAT1 and CsrA-eNRF2. All QPCR samples were processed 72 hrs post-transfection and are the result of at least 3 biological replicates.

FIGS. 12A-12B. Combinatorial recruitment two transcriptional effector with minimal delivery constructs. FIG. 12A. Schematic representation of 2 construct delivery strategy for combinatorial recruitment of two transcriptional effector. FIG. 12B. Relative TTN mRNA expression level following transfection of indicated dual construct for combinatorial recruitment using TTN or non-targeting gRNA. All qPCR samples were processed 72 hrs post-transfection and are the result of at least 3 biological replicates.

### DETAILED DESCRIPTION

Provided herein is a designer multi-stem-loop RNA intermediate effector recruitment platform, called “DRIMER”, that permits robust stochiometric and spatiotemporal control over effector recruitment to targeted human loci when used with CRISPR/Cas systems. DRIMER can recruit up to four different effector domains to human loci. Importantly, these effectors can be functionally distinct and recruited in user-defined combinations. For instance, using the DRIMER system, the inventors demonstrated powerful synergistic effects following the recruitment of multiple synthetic transcription factors and/or epigenetic editors with respect to transcription activation from human promoters. Further, DRIMER-mediated recruitment can be precisely tuned by incorporating chemically controlled riboswitches or optogenetically regulated stem-loops. Overall, the DRIMER platform is a highly programmable CRISPR-based system that allows stoichiometric and combinatorial effector recruitment using designer, structured RNAs for the precise modulation of human gene expression and other genomic activities, which is an attractive capability for a wide range of applications spanning complex epigenome editing as well as cell and gene therapy applications.

These and other aspects of the disclosure are described in detail below.

### I. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

### II. DRIMER Platform

The ability to modulate gene expression in a precise manner is an absolute requirement for controlling biological phenomena to produce therapeutic transcriptional correction, model disease, differentiate cells in a targeted manner, dissect the complex relationships of genetic regulatory functions, and optimize cell therapy-based systems for production of high value biomolecules [PMID: 33336604 PMID: 30513193 PMID: 31730408 PMID: 34147719 PMID: 34354157 PMID: 26383226]. Nuclease deactivated CRISPR-Cas9 (dCas9) mediated precise targeting of transcriptional modulators and epigenetic effectors has revolutionized the field of synthetic transcriptional control. Numerous recruitment strategies have been implemented that utilize dCas9 and associated guide RNA (gRNA) to target gene regulatory elements to achieve precise and tunable modulation of gene transcription. The simplest representation of effector recruitment is using direct protein fusion. Further advancement relies on stem loop binding protein (SLBP) interactions with their cognate stem loops (SLs). Previous efforts were taken to engineer gRNA to incorporate SLs or non-coding RNA sequences for aptamer-based recruitment of SLBP-fused proteins and localization to the genomic locus encoded by the gRNA spacer. The first examples utilized MCP-VP64 fusions which recognized and bound MS2 SLs engineered into the gRNA [PMID: 23907171]. Another early application utilized MS2 SLs with MCP-p65-HSF1 fusions for potent gene activation [PMID: 26317473]. The SAM system was generated by utilizing a VP64 C-terminus fusion to dCas9 and the recruitment of MCP-p65-HSF1 by MS2 elements inserted into the gRNA tetraloops [PMID: 25494202]. In yet another example of gRNA engineering, the MS2 and PP7 SLs were appended to the 3′ end of gRNA. It was observed that expression of both MCP-VP64 and PCP-VP64 fusions result in greater than additive transcriptional activation in yeast. It was also observed that dual SL fusion to a single gRNA coupled with dual recruitment of transcriptional activators resulted in similar activation to the single SL strategy [PMID: 25533786]. These previous examples of gRNA engineered recruitment exemplify the limitations of this strategy. Recruitment potency is extremely limited based on gRNA terminus, linker length and limited tetraloop availability as well as the diversity and number of effectors that can be recruited. Certain gRNA modifications are intolerable negatively impacting gRNA structure which can substantially or completely abrogate the potency of transcriptional modulation [PMID: 25533786, PMID: 27733506]. It remains unclear whether extensive gRNA mediated effector recruitment negatively impacts dCas9 binding affinity, RNP formation, or targeting ability. A recent study also demonstrated an RNA scaffold mediated recruitment strategy wherein two different SLs were situated in the scaffold structure and bound by MCP-dCas9 and PCP-fused effectors (VP64, p65-HSF1, or VPR) through MS2:MCP and PP7:PCP recruitment respectively [PMID: 35792375].

To address the shortcoming of previous approaches, the inventors hypothesized that recruitment utilizing a modular, intermediate, and multi stem loop structured designer RNA (dRNA) could effectively collocate dCas9 and an effector molecule simultaneously without compromising gRNA structure. RNA SLs are important secondary structures that have been shown to direct RNA folding, protect structural stability, play a role in post translational regulation, provide recognition sites for SLBPs, and can serve as a substrate for enzymatic reactions [PMID: 35022230 PMID: 16923806 PMID: 17299129]. These SLs are of particular interest due to their small size coupled with the specificity and affinity in which they are bound by their cognate SLBPs. Many SL:SLBP pairs have been reported with the MS2:MCP and PP7:PCP binding partners being widely used for transcriptional activation and intracellular imaging applications [PMID: 23907171 PMID: 25494202 PMID: 27088723]. Recently, the RAT:VVDLicTCAT cognate binding pair has been used to synthetically control many biological processes including transcription and translation [PMID: 34980910].

Using these stem loops the inventors have built a repertoire of synthetic, structured dRNA which contains multiple and different SLs and acts as an intermediate hub to recruit SLBP fused effector molecules including dCas9, transcriptional modulators, and/or epigenetic effectors. The inventors have tested and optimized the configuration of this platform including the number and organization of SLs incorporated into the dRNA as well as the SLBP fusion strategy to dCas9. This optimized configuration was validated with promoter targeted transcriptional activation of multiple endogenous genes. Further, the inventors have incorporated riboswitch in the dRNA to spatiotemporally control gene expression through chemical mediated cleavage or stabilization of the riboswitch. The inventors have also designed an optically controllable dRNA by incorporating SLs and their cognate SLBP in a light dependent manner. The inventors have also characterized and optimized another bacteria derived orthogonal SL:SLBP pair using the wildtype carbon storage regulator B (CsrB) motivated SL motifs we derived the CsrB derived (CBΔ) SLs which are bound by their cognate CsrA SLBP. The CBΔ:CsrA binding pair mediated recruitment was incorporated into the dRNA. This recruitment pair had not been used previously for CRISPRa applications, is similarly potent to, and considerably more compact than the MS2:MCP, PP7:PCP binding pair. This new binding pair potently activates transcription with many well characterized effectors.

Finally, using multiple and different SLs in a single dRNA, the inventors have shown the combinatorial recruitment of numerous unique transcriptional modulators and epigenetic effectors to genomic loci for enhanced transcriptional activation. Overall, the engineered structured, multi stem loop containing dRNA and the DRIMER platform that the inventors have developed here are functional across programmable CRISPR dCas systems, dCas9 species, and cell lines tested enabling robust, tunable, modular, and controllable transactivation in human cells.

This approach resulted in of a versatile and modular platform for the recruitment of effectors in a user defined manner. Most importantly, the inventors have also engineered a new SL:SLBP recruitment pair which had not been used previously for CRISPRa.

Recent studies have demonstrated that combinatorial recruitment of multiple transcriptional modulators/epigenetic effectors exhibit synergistic modulation of transcription [PMID: 32106616 PMID: 31541098]. Here we have shown that our unique and potent recruitment strategy can effectively colocalize multiple transcriptional modulators and/or epigenetic effectors in a simple user-defined manner by changing the stem loops present in the dRNA. This approach is amenable to stoichiometric effector recruitment through the user-defined number and identity of SLs within a dRNA. The organization of these effectors is also tunable by the orientation individual SLs are located on an individual dRNA. We believe the DRIMER platform has immense potential for the identification of synergy between endogenous proteins in situ. We have observed that the size of an effector size may play a role in recruitment. Specifically, many our SLBP proteins natively operate through homodimer formation which may be sterically hindered through the incorporation of large effector proteins [PMID: 11953318 PMID: 19619561 PMID: 8440248 PMID: 18066080]. Further optimization, such as linker length, can be completed to increase the transcriptional potency of targeting with large effectors.

The DRIMER platform has great potential for metabolic engineered biopharmaceutical production in mammalian cells. Mammalian cells represent the greatest proportion of biopharmaceutical systems due to their ability to perform human-like post translational modifications to therapeutic proteins [PMID: 26383226]. The inventors' system can increase the production of progesterone or testosterone based on the multiplex targeting scheme. This strategy can also be expanded to both activate and repression gene expression in a pathway to maximize production of a target biopharmaceutical. This could be accomplished utilizing two orthogonal DRIMER systems. The stimulus responsive Ribo-DRIMER and Opto-DRIMER show promise for mammalian metabolic engineering with multiplex gene activation through chemical or light control. These applications of the DRIMER platform exemplify the versatility and modularity which can be applied to many diverse research questions.

The DRIMER platform has several modules that can be delivered individually in a user-defined manner to rapidly search a design space and optimize effector number, orientation, and identity to elicit an intended transcriptional modulation. These components can be consolidated for double and all-in-one lentiviral vector delivery. This double lentiviral system was successfully packaged and transduced to enable gene activation. These lentiviral delivery strategies highlight the potential for the DRIMER platform to achieve delivery and targeted transcriptional modulation in therapeutically relevant cells. Overall, the strategy presented here for designing and applying designer structured RNA based SLBP fused effector recruitment has numerous potential applications beyond transcriptional modulation.

### A. CRISPR

CRISPRs (clustered regularly interspaced short palindromic repeats) are DNA loci containing short repetitions of base sequences. Each repetition is followed by short segments of “spacer DNA” from previous exposures to a virus. CRISPRs are found in approximately 40% of sequenced eubacteria genomes and 90% of sequenced archaea. CRISPRs are often associated with Cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and silence these exogenous genetic elements like RNAi in eukaryotic organisms.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

### B. Cas Nucleases

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (~30 base pairs in length), which are then somehow inserted into the CRISPR locus near the leader sequence. RNAs from the CRISPR loci are constitutively expressed and are processed by Cas proteins to small RNAs composed of individual, exogenously derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype Ecoli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

Cas9 is a nuclease, an enzyme specialized for cutting DNA, with two active cutting sites, one for each strand of the double helix. The team demonstrated that they could disable one or both sites while preserving Cas9's ability to locate its target DNA. Jinek et al. (2012) combined tracrRNA and spacer RNA into a “single-guide RNA” molecule that, mixed with Cas9, can find and cut the correct DNA targets and such synthetic guide RNAs are used for gene editing.

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Wang et al. (2013) showed that coinjection of Cas9 mRNA and sgRNAs into the germline (zygotes) generated mice with mutations. Delivery of Cas9 DNA sequences also is contemplated.

The systems CRISPR/Cas are separated into three classes. Class 1 uses several Cas proteins together with the CRISPR RNAs (crRNA) to build a functional endonuclease. Class 2 CRISPR systems use a single Cas protein with a crRNA. Cpf1 has been recently identified as a Class II, Type V CRISPR/Cas systems containing a 1,300 amino acid protein. See also U.S. Patent Publication 2014/0068797, which is incorporated by reference in its entirety.

In some embodiments, the compositions of the disclosure include a small version of a Cas9 from the bacterium Staphylococcus aureus (UniProt Accession No. J7RUA5). The small version of the Cas9 provides advantages over wildtype or full length Cas9. In some embodiments the Cas9 is a spCas9 (AddGene).

C. gRNA

A small guide RNA (sgRNA), or gRNA is an RNA with around 20 nucleotides used to direct Cas9 or dCas9 to their targets. gRNAs contain two major regions of importance for CRISPR systems: the scaffold and spacer regions. The spacer region has nucleotides that are complementary to those found on the target genes, often in the promoter region. The scaffold region is responsible for formation of a complex with (d) Cas9. Together, they bind (d) Cas9 and direct it to the gene(s) of interest. Since the spacer region of a gRNA can be modified for any potential sequence, they give CRISPR systems much more flexibility as any genes and nucleotides with a sequence complementary to the spacer region can become possible targets.

### D. Delivery Systems

Delivery systems for the CRISPR-based systems of the present disclosure include non-viral systems (genetic constructs in liposome, lipid nanoparticles, nanobeads, etc.) or integrative vectors such as retroviral or lentiviral system, or transient delivery systems, such as adeno-associated virus (AAV) or non-integrating lentiviruses. In embodiments, the AAV vector is replication-defective or conditionally replication defective. In embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11 or any combination thereof.

### E. Stem-Loop Binding Proteins

The systems disclosed herein are based on RNA bridge-mediated effector protein recruitment. More specifically, the systems take advantages of various RNA motif/RNA binding protein binding pairs. To this end, a RNA bridge is designed such that a first stem loop (e.g., MS2 operator motif), which specifically binds to a first RNA binding protein (e.g., MS2 coat protein, MCP), is linked a second stem loop, which specifically binds to a second RNA binding protein, where one of the RNA binding proteins is fused to the CRISPR/Cas9 protein or derivatives and the other RNA binding protein is fused to an effector protein. The RNA bridge can then have further stem loops that bind specifically to further RNA binding proteins, which are fused to other effector proteins to bring together combinations of effectors.

As a result, this RNA bridge component of the platform disclosed herein is a designed RNA molecule, which contains the recruiting RNA stem loops for effector recruiting. In this way, recruited-effector protein fusions can be recruited to the target site through their ability to bind to the recruiting RNA motif. Due to the flexibility of RNA scaffold mediated recruitment, a functional monomer, as well as dimer, tetramer, or oligomer could be relatively easy to form near the target DNA or RNA sequence. These pairs of RNA recruiting motif/binding protein could be derived from naturally occurring sources (e.g., RNA phages, or yeast telomerase) or could be artificially designed (e.g., RNA aptamers and their corresponding binding protein ligands). A non-exhausting list of examples of recruiting RNA motif/RNA binding protein pairs that could be used in the described systems are summarized in Table 1.

TABLE 1
Exemplary Stem Loop: Stem-Loop Binding Protein Pairs

| Stem Loop | Stem-Loop Binding Protein |
|---|---|
| Telomerase Ku binding hairpin | Ku |
| Telomerase Sm7 binding motif | Sm7 |
| MS2 phage operator stem-loop | MS2 Coat Protein (MCP) |
| PP7 phage operator stem-loop | PP7 coat protein (PCP) |
| SfMu phage Com stem-loop | Com RNA binding protein |
| CsrB-SL | E. coli Carbon storage regulator A (CsrA) |
| ribonucleic antiterminator (RAT) sequence | LicT from Bacillus subtilis |

Exemplary sequences for some of the above binding pairs are listed below.

MS2 phage operator stem loop:
(SEQ ID NO: 1)
5′-GCGCACAUGAGGAUCACCCAUGUGC-3′
MS2 coat protein:
(SEQ ID NO: 2)
MASNFTQFVLVDNGGTGDVTVAPSNFANGIAEISSNSRSQAYKVTCSVRQ
SSAQNRKYTIKVEVPKGAWRSYLNMELTIPIFATNSDCELIVKAMQGLLK
DGNPIPSAIAANSGIY
PP7 phage operator stem loop:
(SEQ ID NO: 3)
5′-AUAAGGAGUUUAUAUGGAAACCCUUA-3′
PP7 coat protein (PCP):
(SEQ ID NO: 4)
MSKTIVLSVGEATRTLTEIQSTADRQIFEEKVGPLVGRLRLTASLRQNGA
KTAYRVNLKLDQADWDCSTSVCGELPKVRYTQVWSHDVTIVANSTEASRK
SLYDLTKSLVATSQVEDLVVNLVPLGR
SfMu Com stem loop:
(SEQ ID NO: 5)
5′-CUGAAUGCCUGCGAGCAUC-3′
SfMu Com binding protein:
(SEQ ID NO: 6)
MKSIRCKNCNKLLFKADSFDHIEIRCPRCKRHIIMLNACEHPTEKHCGKR
EKITHSDETVRY
ribonucleic antiterminator (RAT) stem loop:
(SEQ ID NO: 7)
5′-AUUGUUACUGCUACGGCAGGCAAAA-3′
LicT protein from Bacillus subtilis:
(SEQ ID NO: 8)
KIAKVINNNVISVVNEQGKELVVMGRGLAFQKKSGDDVDEARIEKVFTLD
NKDVS
CsrB stem loop:
(SEQ ID NO: 12)
5′-GAGUCAGACAACGAAGUGAACAUCAGGAUGAUGACACUUCUGCAGGA
CACACCAGGAUGGUGUUUCAGGGAAAGGCUUCUGGAUGAAGCGAAGAGGA
UGACGCAGACGCGUUAAAGGACACCUCCAGGAUGGAGAAUGAGAACCGGU
CAGGAUGAUUCGGUGGGUCAGGAAGGCCAGGGACACUUCAGGAUGAAGUA
UCACAUCGGGGUGGUGUGAGCAGGAAGCAAUAGUUCAGGAUGAACGAUUG
GCCGCAAGGCCAGAGGAAAAGUUGUCAAGGAUGAGCAGGGAGCAACAAAA
GUAGCUGGAAUGCUGCGAAACGAACCGGGAGCGCUGUGAAUACAGUGCUC
CCUUUUUUUAUU-3′
CBΔ type 1-SL:
(SEQ ID NO: 9)
5′-GGCACAAGGAUGUGCC-3′
CBΔ type 2-SL:
(SEQ ID NO: 10)
5′-GCACAAGGAUGUGC-3′
E. coli Carbon storage regulator A (CsrA):
(SEQ ID NO: 11)
LILTRRVGETLMIGDEVTVTVLGVKGNQVRIGVNAPKEVSVHREEIYQRI
QAEKSQQSSY

The RNA bridge molecule may include one or more modifications. Such modifications may include inclusion of at least one non-naturally occurring nucleotide, or a modified nucleotide, or analogs thereof, Modified nucleotides may be modified at the ribose, phosphate, and/or base moiety. Modified nucleotides may include 2′-O-methyl analogs, 2′-deoxy analogs, or 2′-fluoro analogs. The nucleic acid backbone may be modified, for example, a phosphorothioate backbone may be used. The use of locked nucleic acids (LNA) or bridged nucleic acids (BNA) may also be possible. Further examples of modified bases include, but are not limited to, 2-aminopurine, 5-bromo-uridine, pseudouridine, inosine, 7-methylguanosine. These modifications may apply to any component of the CRISPR systems. In a preferred embodiment these modifications are made to the RNA components, e.g. the guide RNA sequence or the bridge sequence.

### F. Effector Proteins

The effector component comprises an activity portion, i.e., an effector domain. In some embodiments, the effector domain comprises the naturally occurring activity portion of a non-nuclease protein. In other embodiments, the effector domain comprises a modified amino acid sequence (e.g., substitution, deletion, insertion) of a naturally occurring activity portion of a non-nuclease protein. The effector domain has an enzymatic activity. Examples of this activity include transactivation activity, deamination activity, methyltransferase activity, demethylase activity, DNA repair activity, DNA damage activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, DNA methylation, histone acetylation activity, or histone methylation activity. In some embodiments, the effector has a transcriptional activation domain selected from the group consisting of VP64, eN3×9, MSN, p65, HSF1, VP16, MyoD1, RTA, SET7/9, VPR, histone acetyltransferase p300, an hydroxylase catalytic domain of a TET family protein (e.g., TET1 hydroxylase catalytic domain), LSD1, CIB1, AD2, CR3, EKLF1, GATA4, PRVIE, p53, SP1, MEF2C, TAX, and PPARγ.

### III. Formulation and Administration

The present disclosure provides pharmaceutical compositions. Such compositions comprise a prophylactically or therapeutically effective amount of an agent, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the agent, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Validation and Selection of CRISPR/dCas9 and dRNA Based Gene Activation Platform DRIMER

The programmable, precisely tunable, and robust control of gene expression has been a long-term interest in the field of biomedical science and cell/gene therapy applications. Programmable and specific control over aberrant gene expression and modulating transcription can help to elucidate biological phenomena in healthy and therapeutically important mammalian cells. Zinc finger, TALE and CRISPR-dCas9 based epigenome editing platform has the power to recruit specified effector protein to a user defined target cis-regulatory element on the genome. However, using these previously characterized tools, only limited number of effector proteins could be recuited. Here, using multi stem loop dRNA based recruitment platform, the inventors could recruit 4 different types of effector proteins into a target loci. The DRIMER platform can be utilized for metabolic engineering of the steroidogenesis pathway using multiplexed gRNA delivery. The inventors have designed a novel platform for localization of proteins with gRNA-targeted CRISPR proteins. Importantly, “designer RNA” (dRNA) which are engineered RNA molecules with various stem-loops (SLs) that act as a bridge between the CRISPR and effector proteins. dRNA SLs are bound by their cognate stem-loop binding proteins (SLBPs).

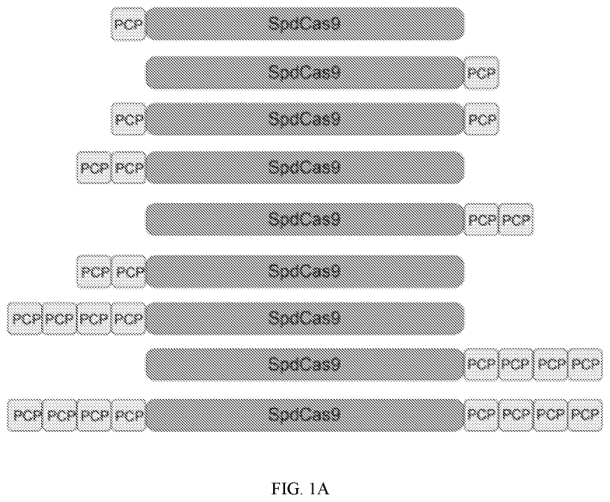

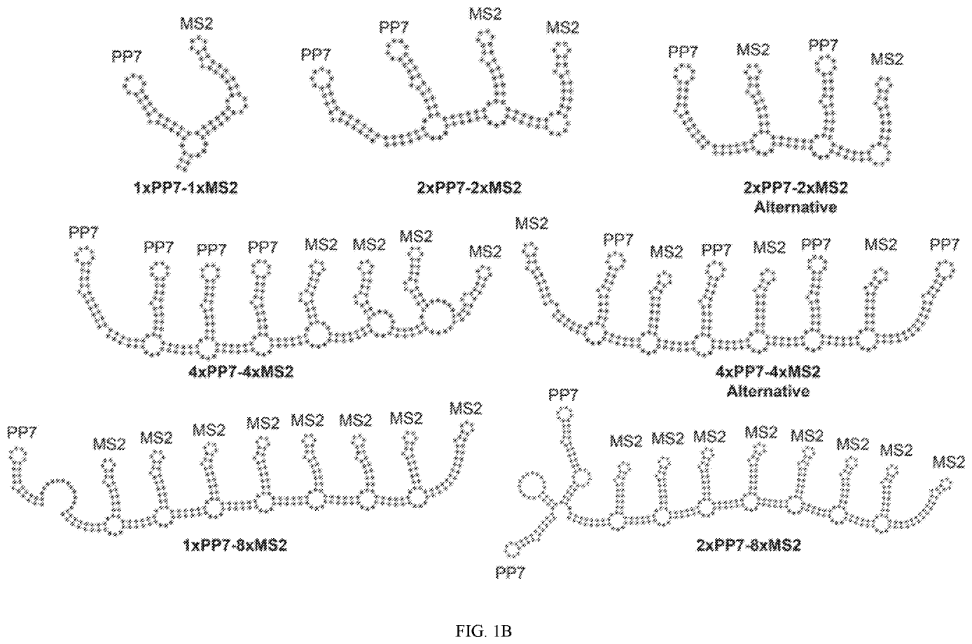

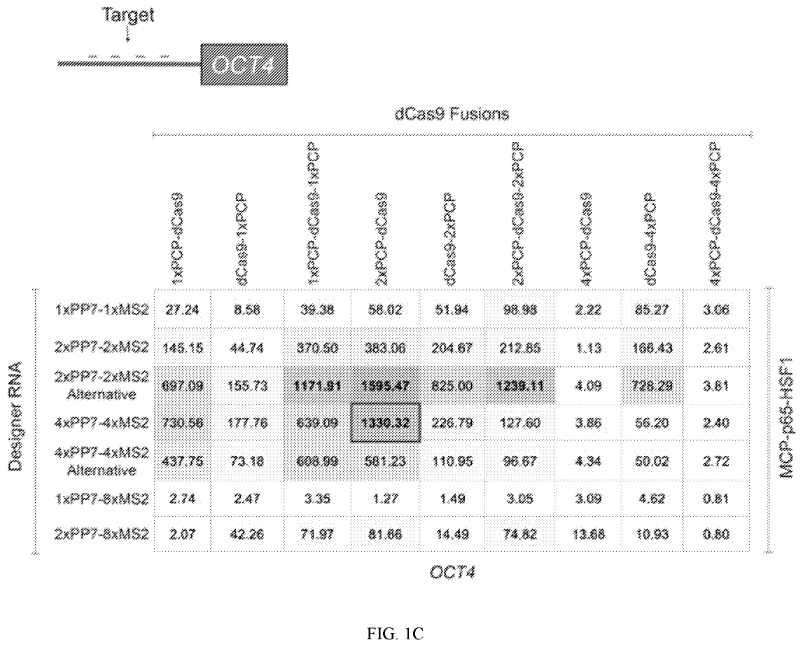

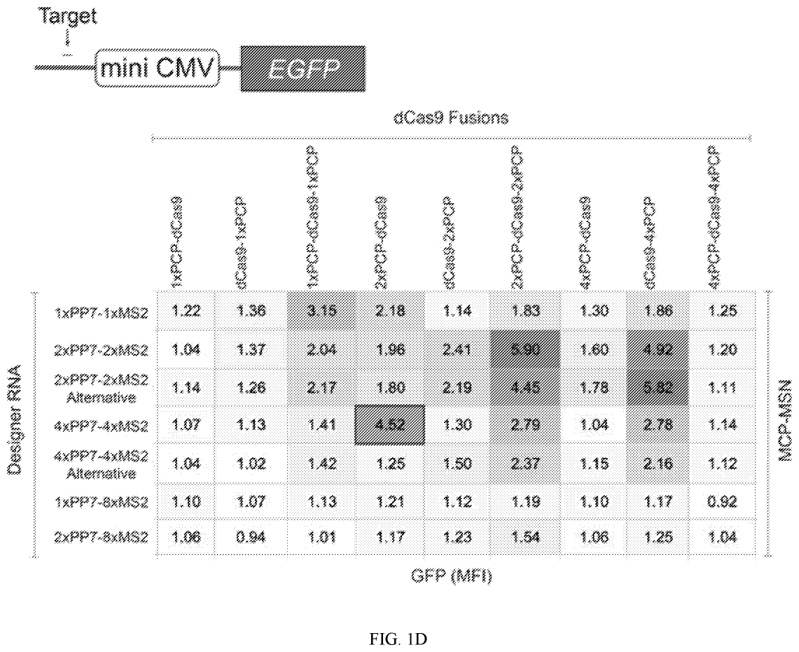

Initial optimization experiments were carried out to identify the system characteristics resulting in the greatest potency and flexibility for future applications. These characteristics included dCas9 SLBP fusion number (0×, 1×, 2×, and 4×) and orientation (N, C, or N and C terminus) which represented 9 possible permutations (FIG. 1A). Similarly, 7 dRNA variants were constructed consisting of different numbers (1×, 2×, 4×, or 8×) of 2 orthogonal SLs (FIG. 1B). These dRNA were constructed to bridge dCas9 and SLBP-fused effector domains requiring high concentration in the nucleus, motivating the use of an RNA polymerase III 7SK promoter driving its expression. For these initial screen and optimization experiments, the inventors utilized the highly validated and widely used MS2:MCP and PP7:PCP cognate binding pairs. The inventors constructed 7 different structured dRNA variants consisting of different numbers of PP7 and MS2 stem-loops as well as the SL organization (FIG. 1B). The MCP protein was fused to MSN or p65-HSF1 for transcriptional activation of a synthetic testbed and an endogenous gene respectively. The initial optimization screen targeted a previously validated HEK293T fluorescent CRISPRa testbed cell line with 70 permutations of the system components were co-transfected including dCas9/PCP fusions, PP7 and MS2 containing dRNA variants, MCP-MSN, and testbed targeting gRNA [PMID: 36162812]. The results clearly demonstrated that many of these dCas9 and RNA binding protein PCP fusions and dRNA variant combinations were highly potent at activating EGFP expression from targeting this synthetic testbed (FIG. 1D). An additional validation screen was performed targeting an endogenous gene, where the inventors co-transfected all system component permutations along with previously characterized transcriptional activator MCP-p65-HSF1 and gRNA targeting endogenous OCT4 promoter into wildtype HEK293T cells finding similar observations to the initial testbed screen (FIG. 1C). From these initial optimization experiments, the inventors selected 2×PCP-dCas9 N-terminus fusion and the 4×PP7-4×MS2 dRNA variant for future experimentation. Of note, several combinations showed comparable gene activation both from synthetic and endogenous targeting. The selection of the 2×PCP-dCas9 and 4×PP7-4×MS2 dRNA configuration was motivated by the following factors i) the (2×PCP) fusion was potent yet relatively compact, ii) a 2×PCP fusion to made directly to the N-terminus of dCas9 was more potent than the C-terminus fusion and was amenable to future C-terminus direct fusions, iii) the tandem 4×PP7-4×MS2 dRNA was potent in both synthetic and endogenous testbed than the alternating 4×PP7-4×MS2 configuration and is flexible to future applications. Overall, this platform shows robust and efficient gene activation. The inventors have named this system the designer RNA intermediated effector recruitment (DRIMER) platform.

### Example 2—DRIMER is a Modular and Portable System for Targeted Transcriptional Activation

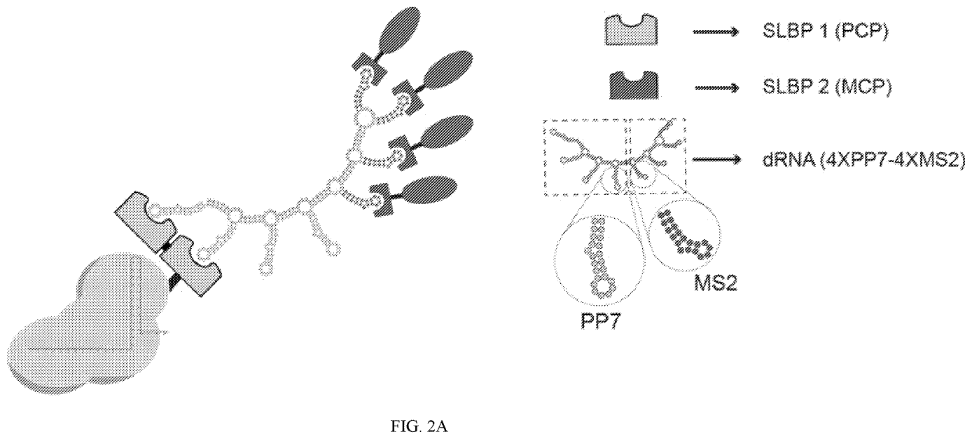

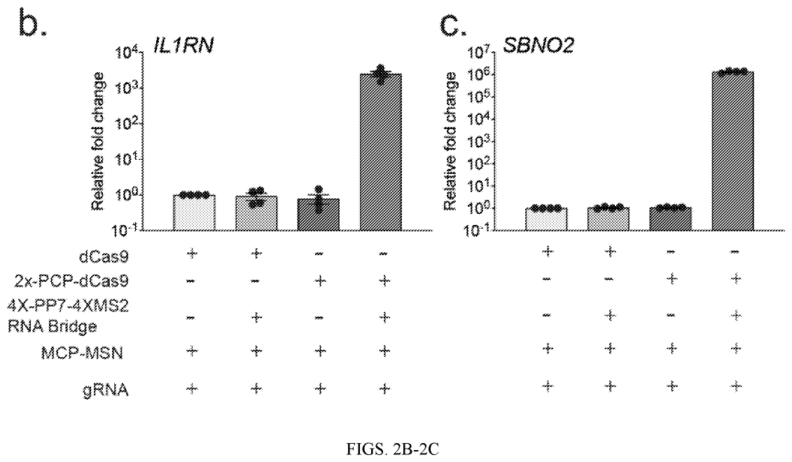

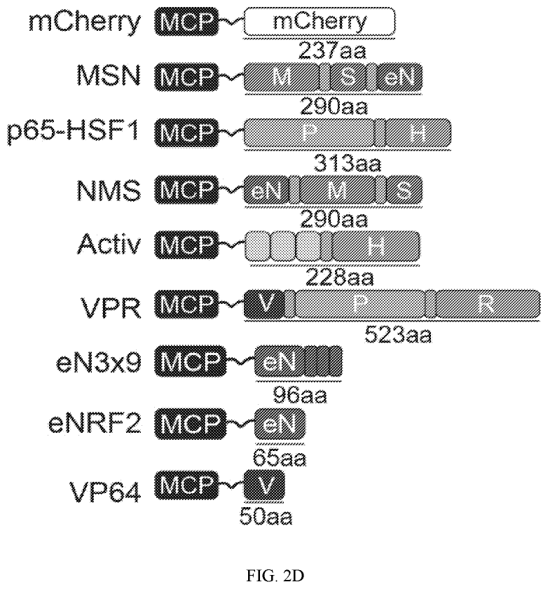

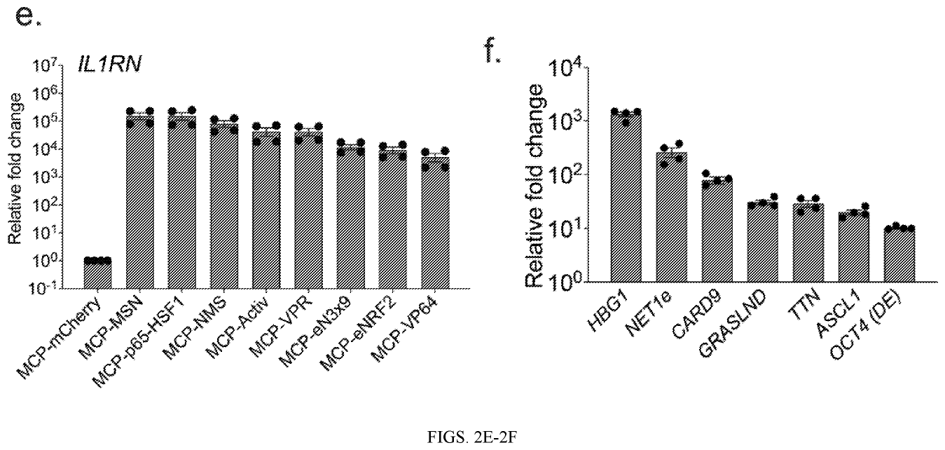

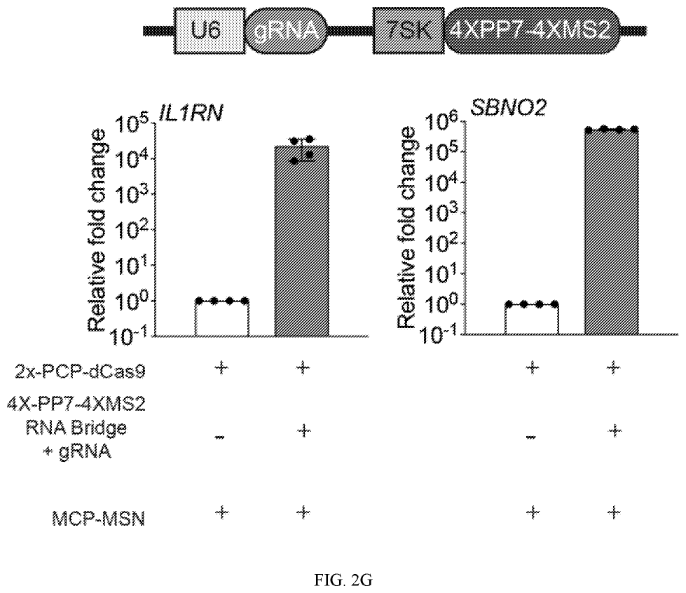

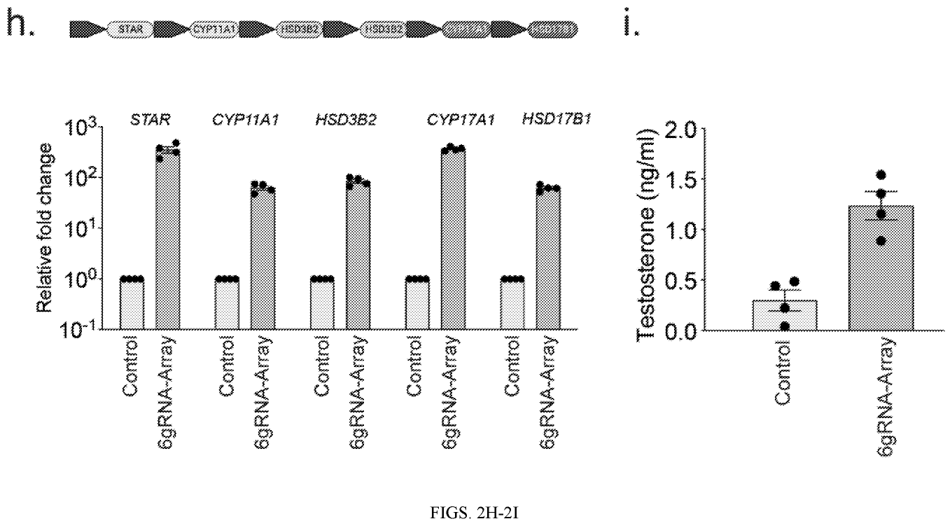

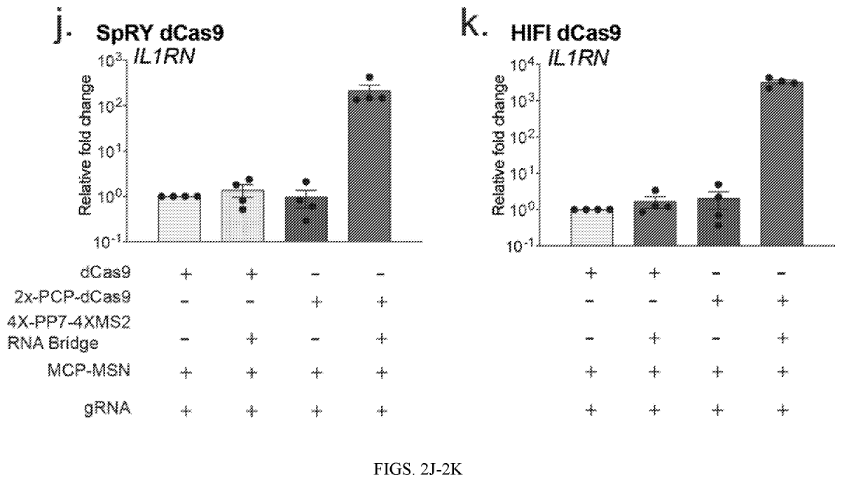

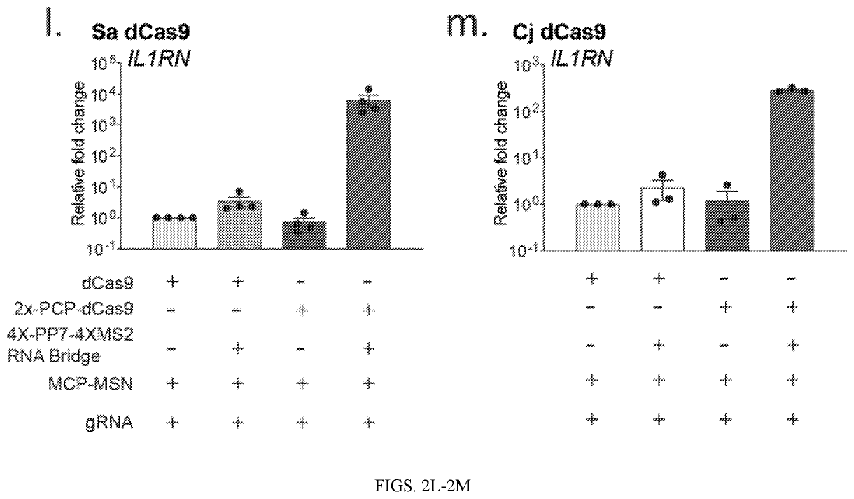

After identifying the optimal DRIMER system architecture (FIG. 2A), the inventors validated selected DRIMER platform mediated gene activation in two endogenous genes IL1RN and SBNO2 (FIG. 2A). Further, the inventors studied the modularity of the DRIMER system by recruiting a handful of MCP fused synthetic transcriptional activators to the promoter region of the IL1RN locus (FIG. 1D). The inventors also observed the potency of the DRIMER platform to activate transcription of various types of RNA (mRNA, lncRNA, and eRNA) within the mammalian genome when targeting promoter or enhancer regions (FIG. 2F). The inventors also observed that gRNA and dRNA could be expressed from a single plasmid backbone using two different potent polIII promoter (FIG. 2G). Further, to activate 5 lowly expressed (STAR, CYP11A1, HSD3B2, CYP17A1, and HSD17B1) endogenous genes in HEK293T cells, modulating cholesterol to testosterone production, the inventors constructed a gRNA array consisting of 6 gRNAs targeting all 5 genes and used along with DRIMER and demonstrated simultaneous activation of all 5 genes (FIG. 2H) and subsequently testosterone production (FIG. 2I).

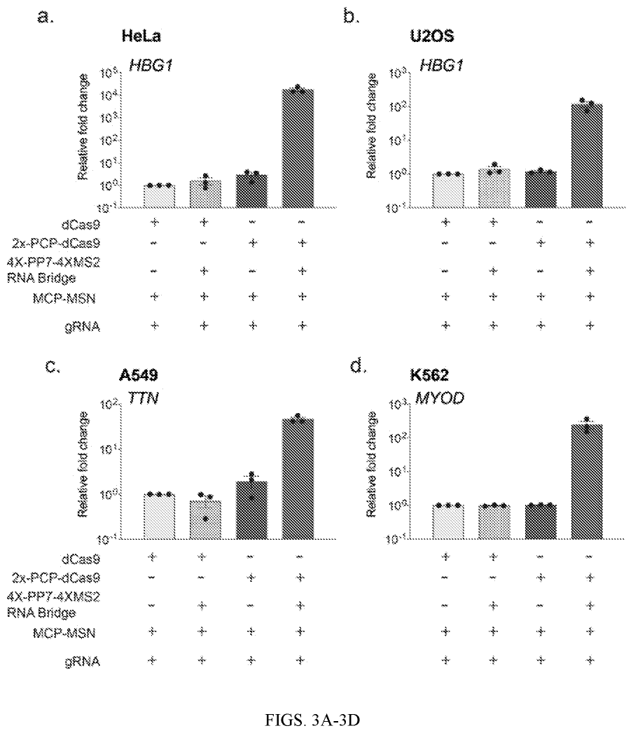

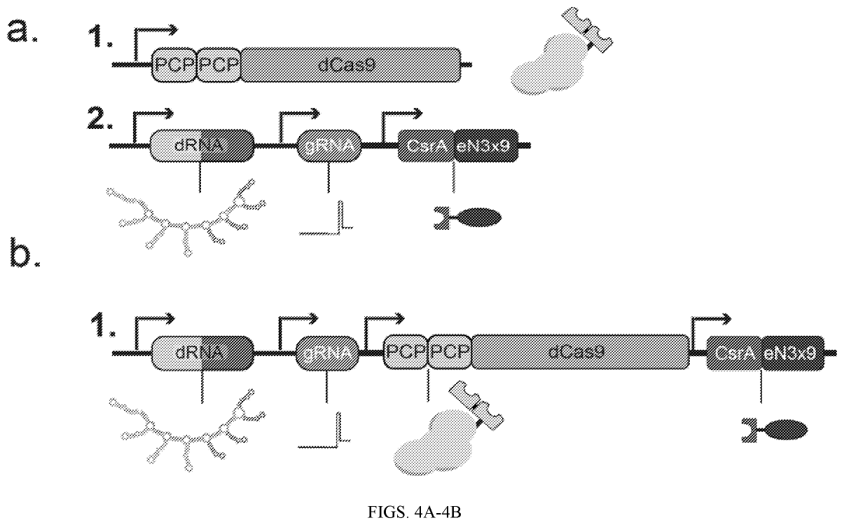

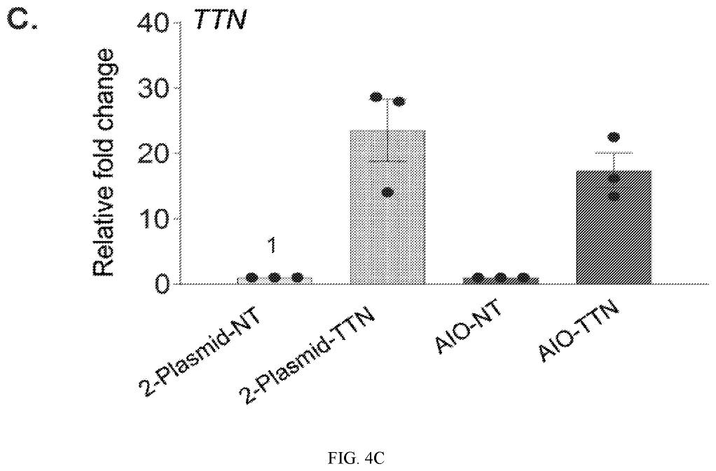

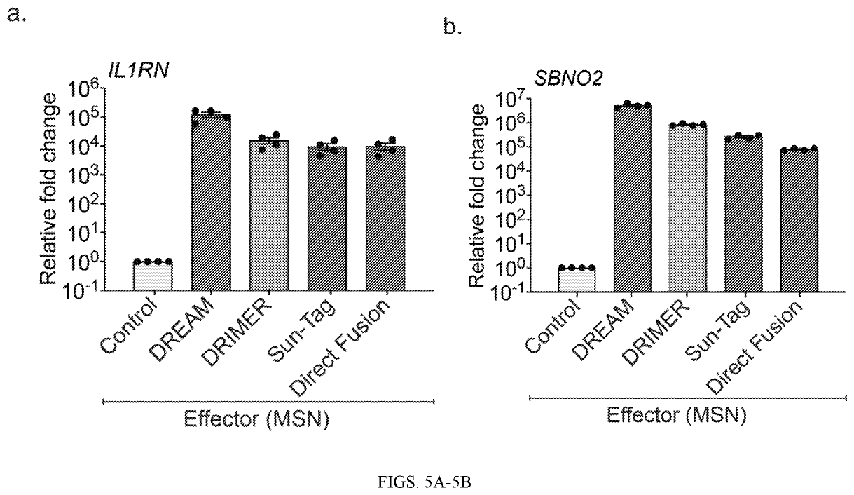

For broader application of DRIMER, the DRIMER platform is portable to engineered SpdCas9 variants SpRY dCas9 and HIFI dCas9 (FIGS. 2J-2K) for endogenous IL1RN gene activation. Further, these data show small orthogonal dCas9 proteins SadCas9 and CjdCas9 could also be used to target endogenous genes IL1RN (FIGS. 2J-2M). The DRIMER platform is also portable to type I CRISPR systems. The DRIMER platform potently activates transcription when targeting the promoter region of endogenous genes in four additional cell lines (FIGS. 3A-3D). Finally, efforts were made towards minimizing the number of constructs necessary for the delivery of all DRIMER components and DRIMER components could be delivered using all-in-one (AIO) plasmid constructs (FIGS. 4A-4C). In the end, the inventors have compared the gene activation potential of RNA aptamer-based DREAM, antigen-antibody based Sun-tag system and direct fusion based programmable gene activation platforms using tripartite transcriptional activator MSN, and these data demonstrated DRIMER based gene activation is better than sun-tag based or direct fusion-based gene activation platform at least when transcriptional activator MSN was used (FIG. 5). Overall, these data clearly demonstrate robustness, modularity, and portability of DRIMER platform in activation of human genes.

### Example 3—DRIMER Variants Allow Metabolic Engineering in a Stimulus Dependent Manner

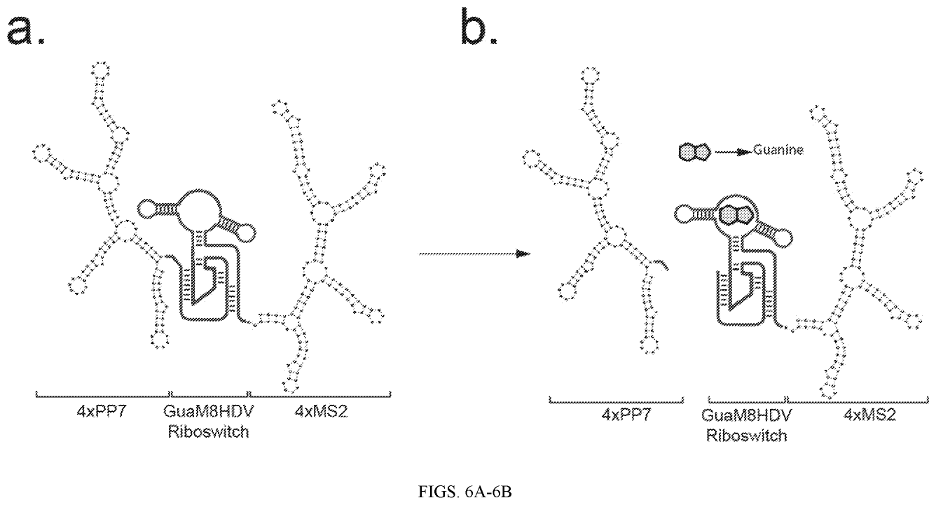

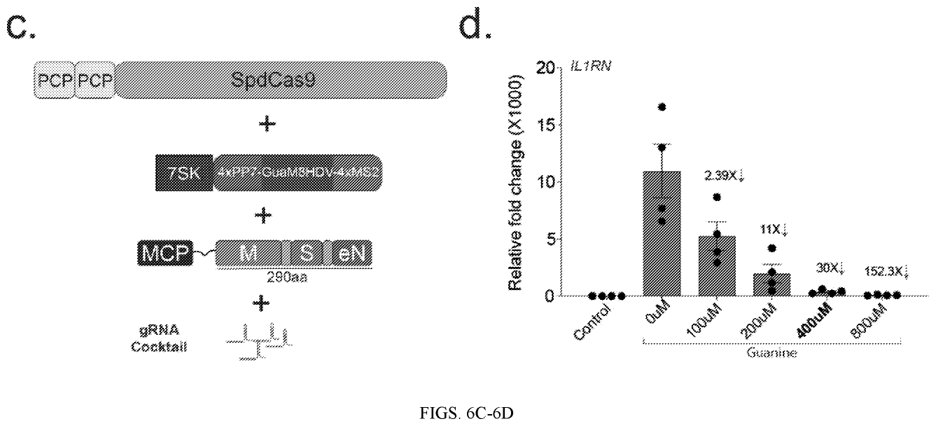

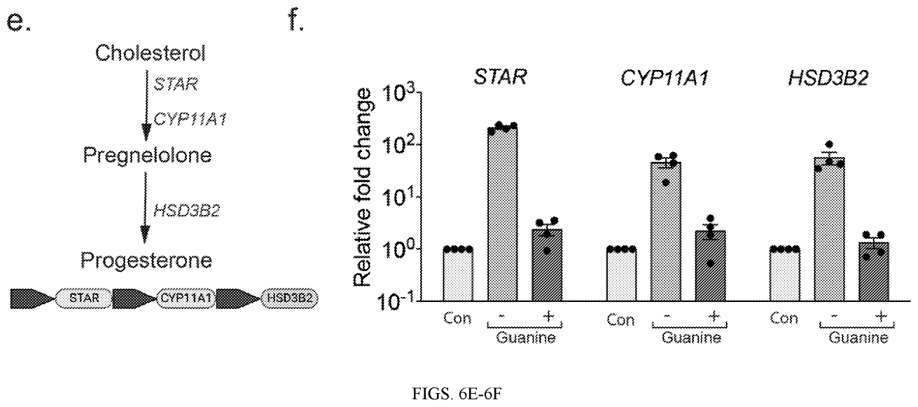

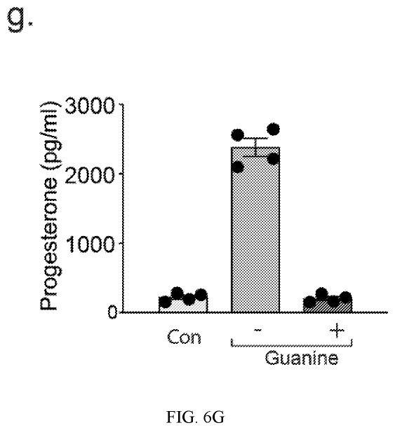

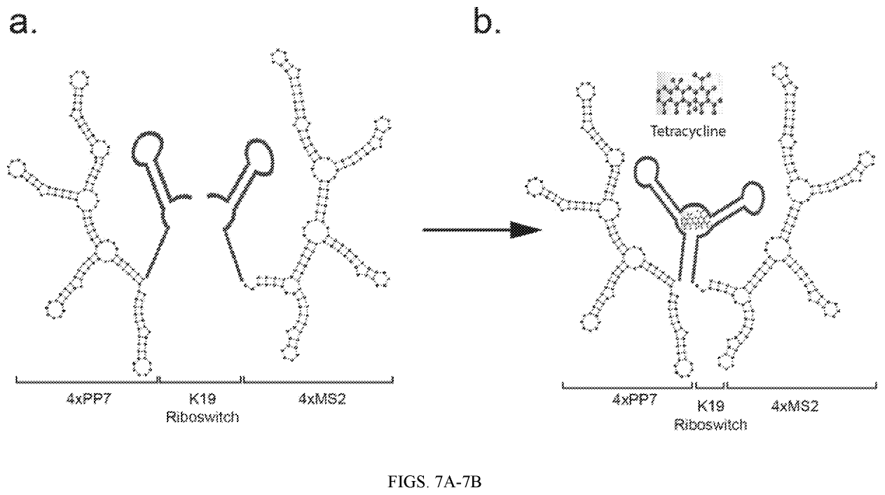

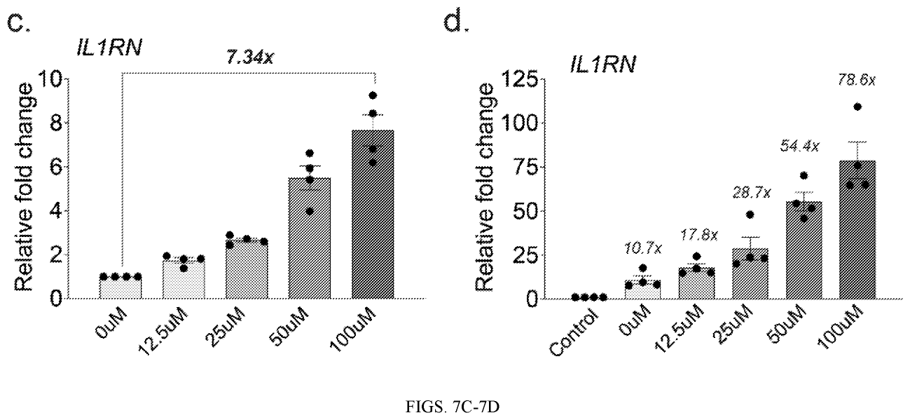

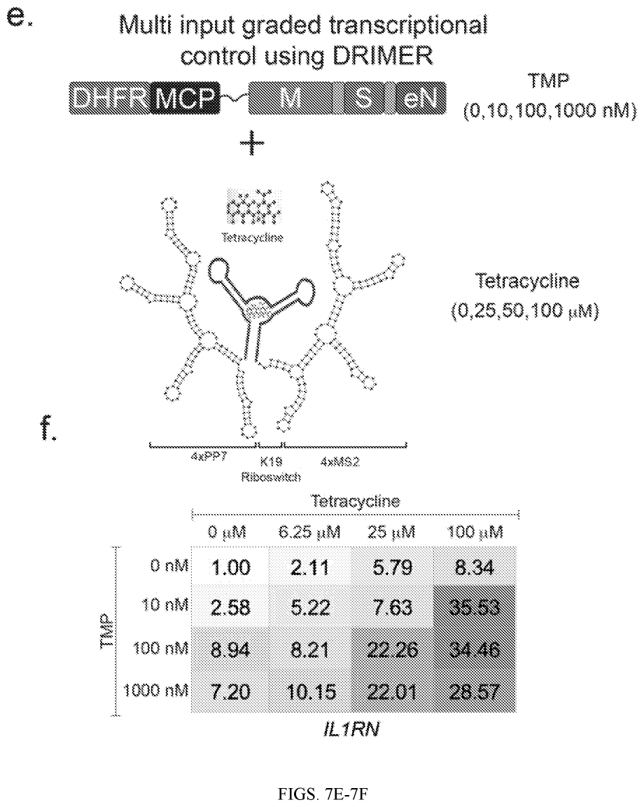

Previous engineering has enabled the chemical control of CRISPR-Cas-based activation through small molecule dimerization and miRNA agonist destabilization [PMID: 30710419 PMID: 35792375]. Chemical control over gene expression has many applications from therapeutics to biomanufacturing in prokaryotes and eukaryotes [PMID: 37069267 PMID: 17933507 PMID: 31679824]. First, the inventors explored the chemical control of effector recruitment using incorporation of self-stabilized and small molecule/biomolecule induced cleavable OFF-Riboswitches or self-cleavable and small molecule/biomolecule stabilized ON-Riboswitch into the dRNA region separating two orthogonal tetra-SLBP RNA structures (FIGS. 6, 7).

Incorporation of the Guanine dependent cleavable GuaM8HDV riboswitch allows interruption of effector recruitment in a guanine concentration dependent manner (FIGS. 6A-6D). To further explore the utility of this guanine regulated platform, the inventors successfully used this riboswitch containing dRNA in combination with single vector multiplex gRNA (harboring 4 gRNA targeting 3 metabolic genes) to regulate the expression of metabolic enzymes in human progesterone biosynthesis pathway (FIGS. 6E-6G). Conversely, incorporation of a tetracycline dependent ON-riboswitch K19 confers positive regulatory control in a dose dependent manner (FIGS. 7A-7D). ON-riboswitch K19 based DRIMER platform shows leakiness and we have introduced small molecule stabilized DHFR mediated control of transcriptional activator to bypass the induction of genes even in the absence of tetracycline (FIGS. 7E-7F).

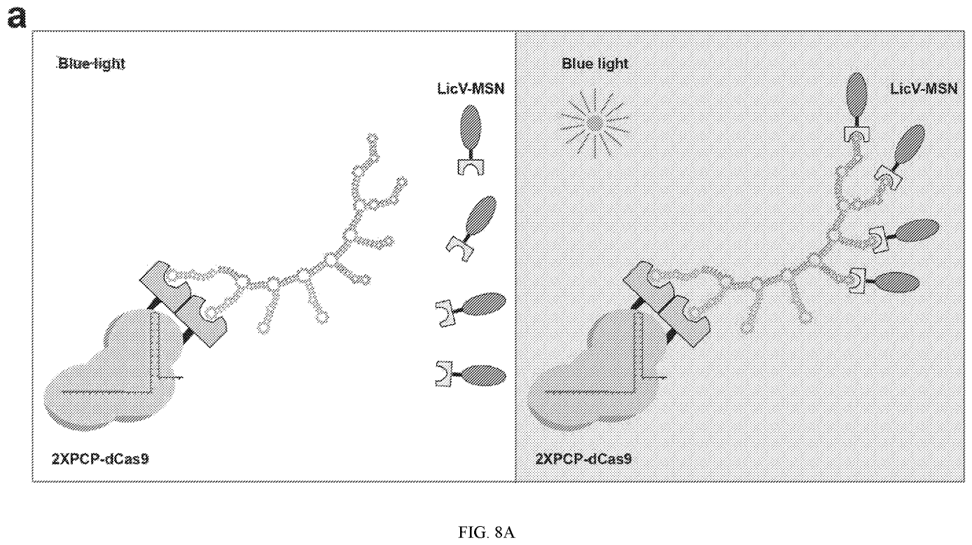

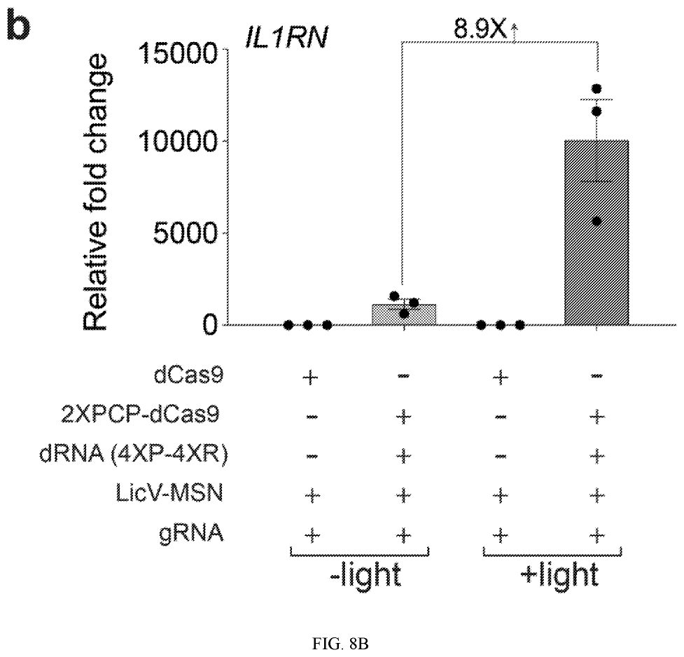

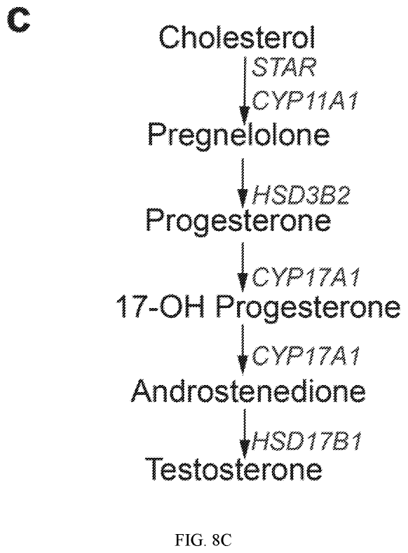

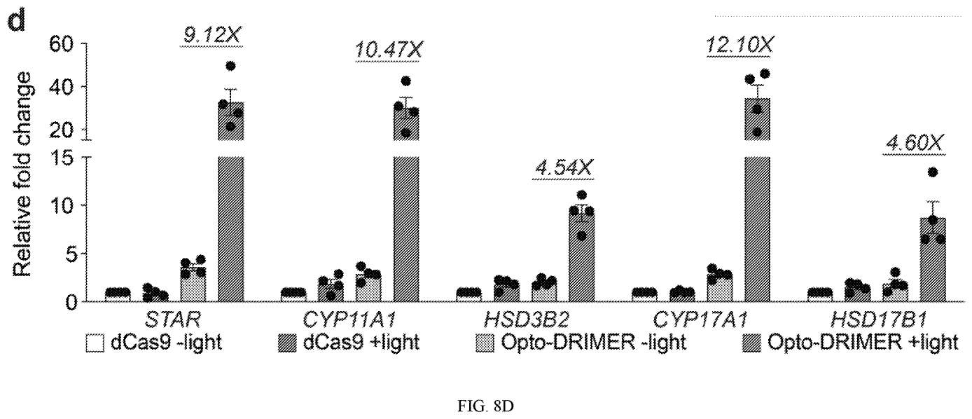

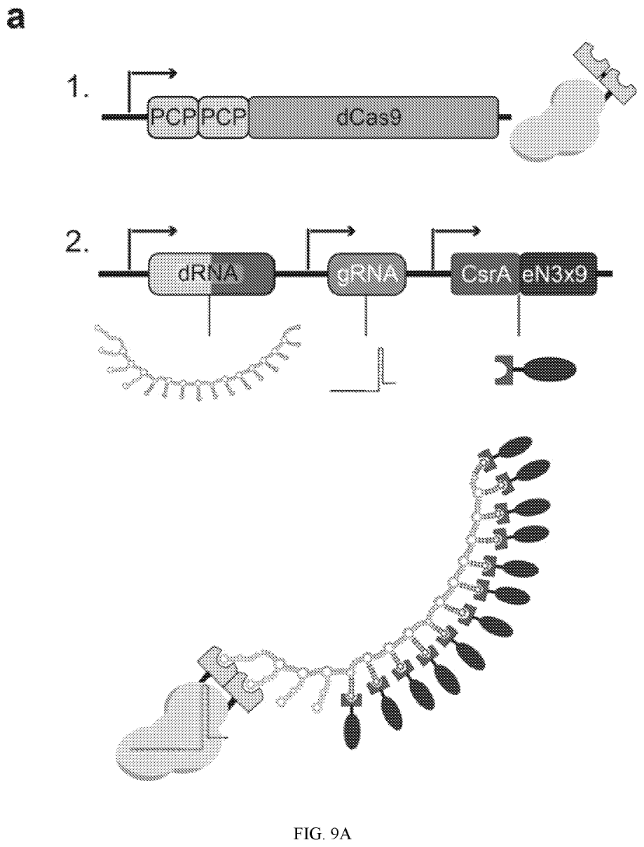

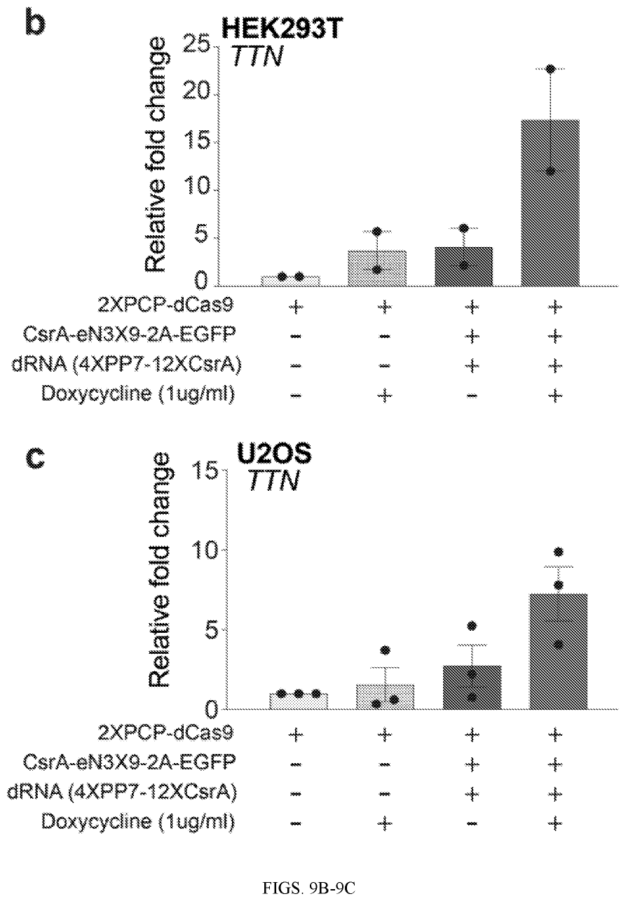

Small molecule contamination presents a risk to downstream processing of biologics and recombinant protein production and purification strategies [PMID: 34336550]. Therefore, the inventors explored the optogenetic control of effector recruitment. This was accomplished by the construction of a dRNA which harbored 4×PP7 and 4×RAT. The inventors fused the previously described LicT-VVD (LicV) SLBP module to the MSN effector. This fusion was demonstrated to bind the RAT SL in the presence of blue light to regulate target gene expression (FIG. 8A) [PMID: 34980910]. Using this platform, first the inventors have demonstrated successful induction IL1RN expression in presence of blue light. Further, using optogenetically controlled DRIMER platform, the inventors have shown increased biosynthesis of testosterone with a multiplex gRNA expression plasmid targeting (harboring 6 gRNA targeting 5 genes) mammalian steroidogenesis pathway (FIGS. 8C and 8D). Doxycycline inducible expression of 4×PCP-dCas9 and effector fusion protein (Csr-eN3×9) was achieved utilizing the 4×PP7-12×CBA dRNA in 2 lentiviral vector plasmids. Dox inducible activation of TTN was observed with single gRNA targeting in HEK293T and U2OS cells with constructs delivered via transient transfection and lentiviral transduction respectively (FIG. 9).

### Example 4—The Generation of a Biologically Inspired Miniature and Potent SL:SLBP Pair

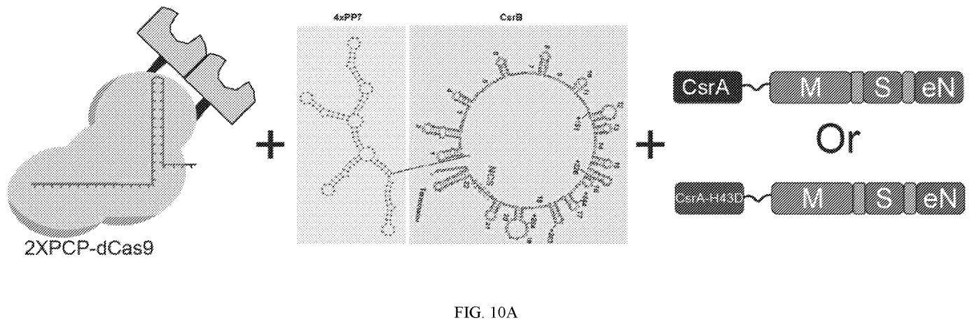

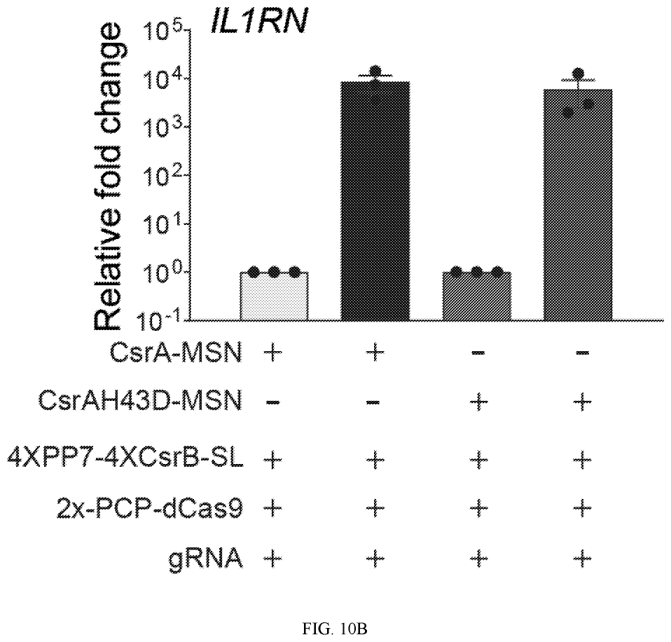

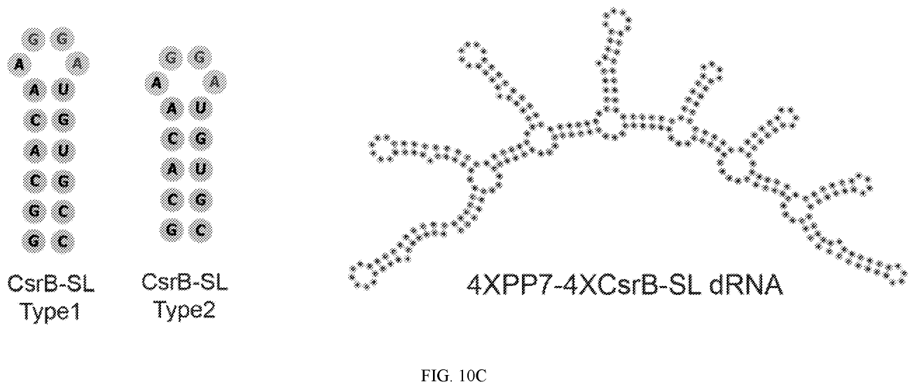

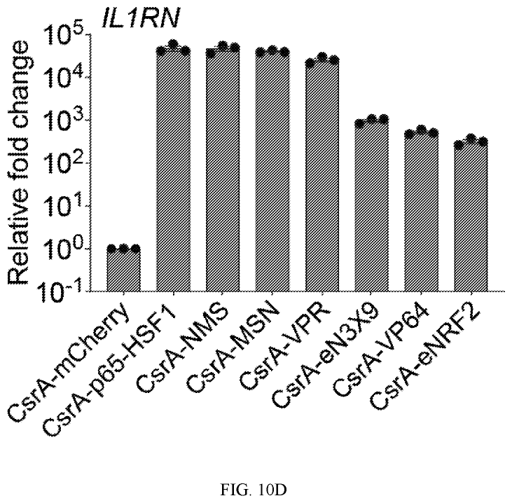

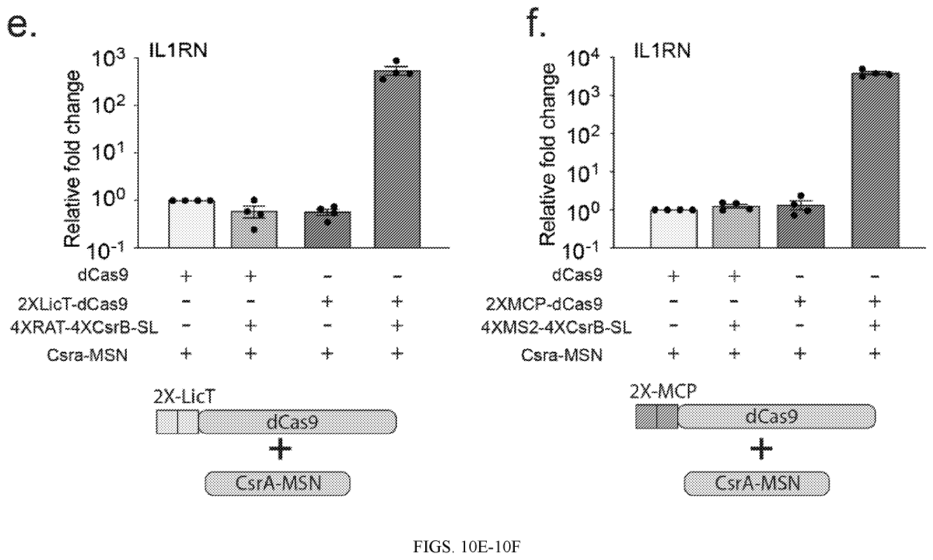

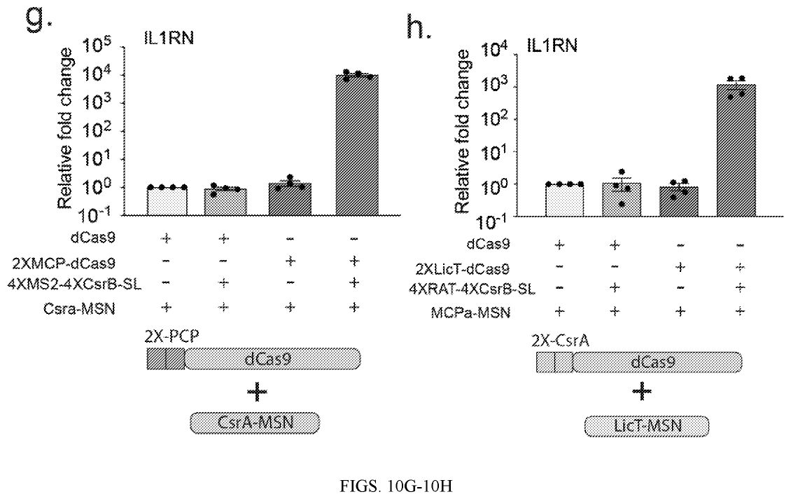

Many examples of SL:SLBP pairs have been identified from viral and bacterial systems. The carbon storage regulator A (CsrA) binding protein is known to post transcriptionally regulate RBS binding site availability or RNA stability in Escherichia coli regulates translation in numerous genes [PMID: 11567002 PMID: 12867454]. CsrA homo dimers have been shown to bridge nearby RNA targets with binding stability associated with distance between these elements [PMID: 19619561]. CsrA availability is regulated with the agonist CsrB RNA. CsrB is a highly structured molecule with numerous CsrA binding elements which can tightly bind ~18 (9 homodimer) CsrA proteins. CsrA homodimers have been hypothesized to interact with two elements with functional domains located on opposite sides of the homodimer complex [PMID: 16923806]. The inventors initial experiment utilized a dRNA design of 4×PP7-1×CsrB-WT and indicated that both WT CsrA and a rationally engineered H43D CsrA variant potently induce transcription when fused to the transcriptional activator MSN and targeted to the promoter region of the IL1RN locus (FIG. 10B). These results supported the ability of CsrB elements to strongly recruit both CsrA variants resulting in similar levels of transcriptional activation. 2 CsrB-WT binding elements were designed to mimic the structure of CsrB binding elements and were named CBΔ types 1 and 2. The engineered SLs were then ported into dRNA with the 4×PP7-4×CBΔ configuration (FIG. 10C). A handful of transcription activators effectors previously tested were fused to CsrA and their activation potency tested. Strikingly, CBΔSL:CsrA-effector fusions also induce high level of transcription from endogenous IL1RN promoter (FIG. 10D). Further, a CsrA fused transcriptional activator could be recruited to other SL binding protein (LicT/MCP/PCP) fused dCas9 for gene activation (FIGS. 10E-10G). On the other hand, CsrA could be fused to dCas9 (2×CsrA-dCas9) to recruit MCP/PCP or LicT fused transcriptional activator to gene activation (FIGS. 10H-10J). Overall, the inventors have engineered an potent and compact SL:SLBP pair for many future biotechnological applications.

### Example 5—The Modular Nature of DRIMER Facilitates the Identification of Synergy Between Effector Proteins

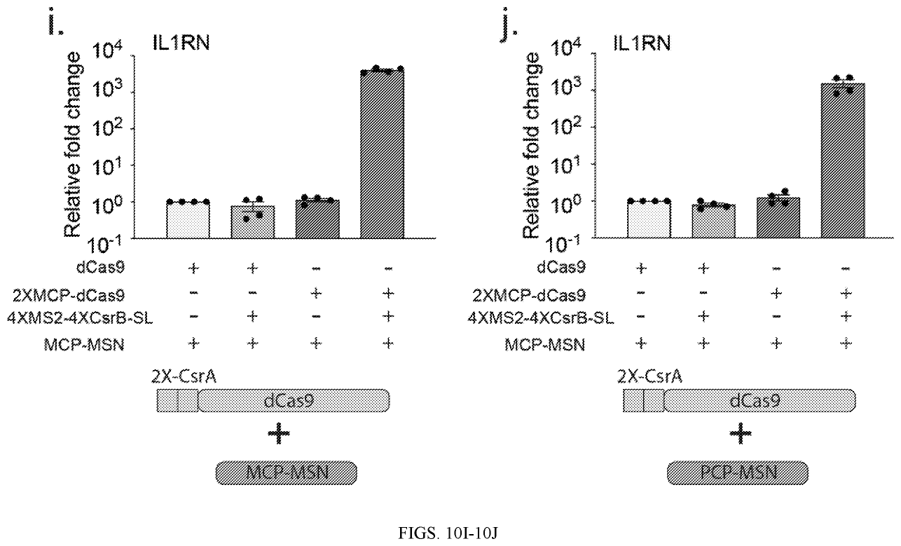

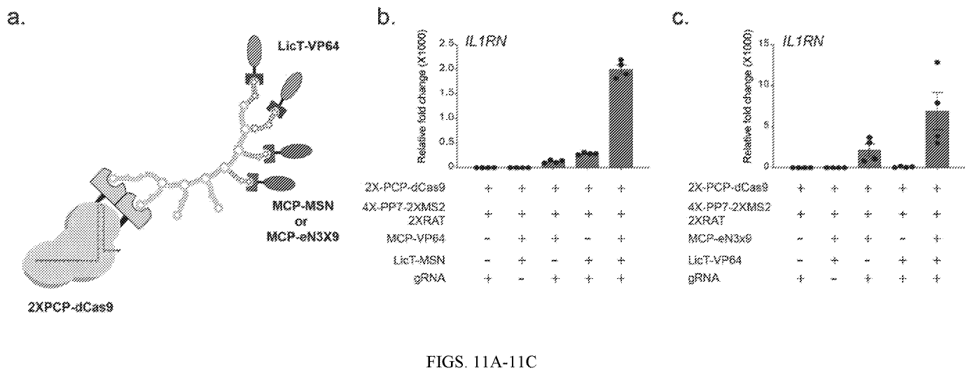

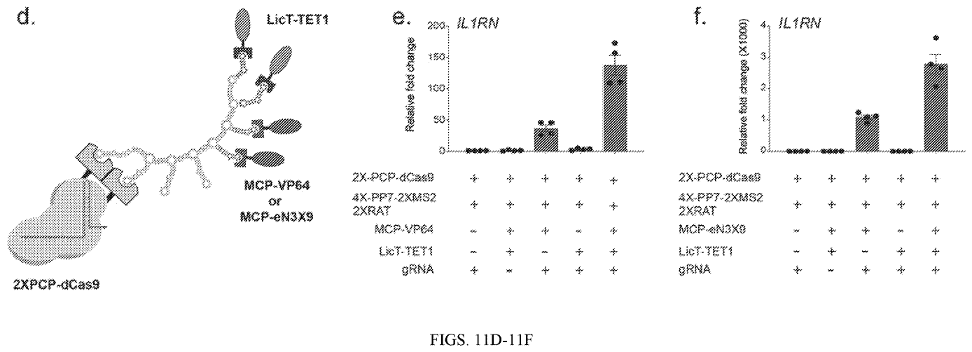

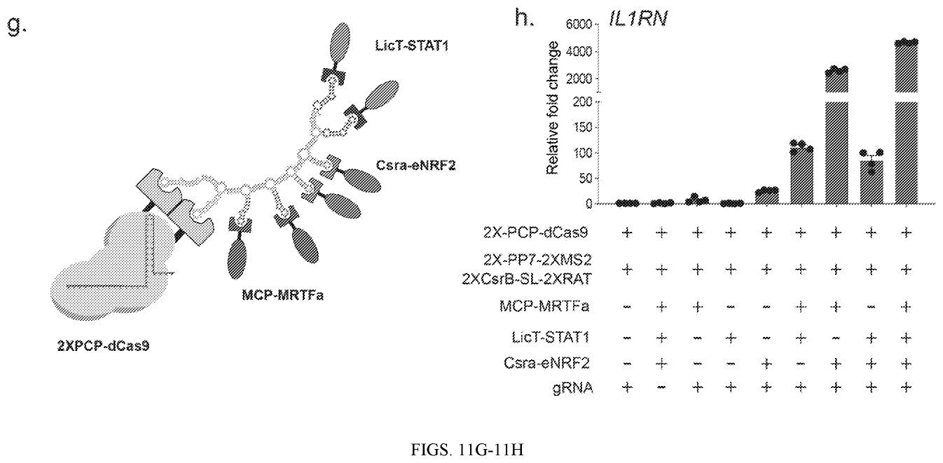

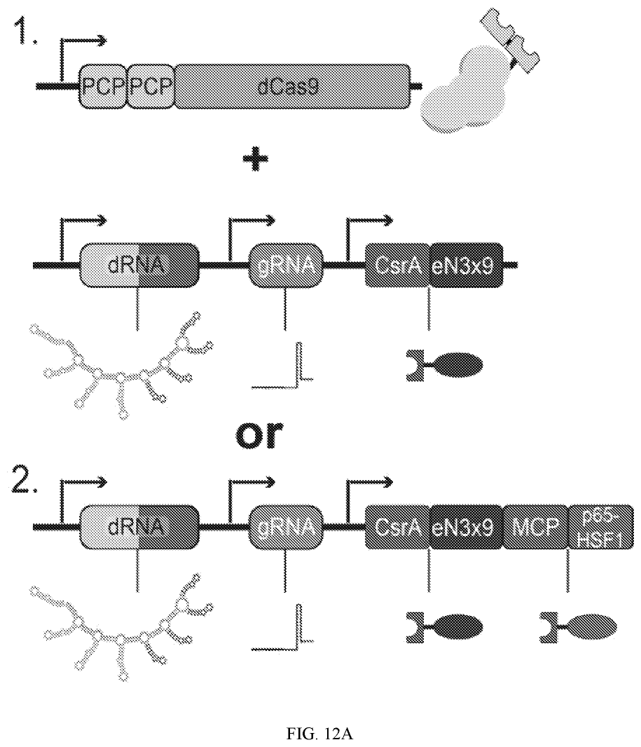

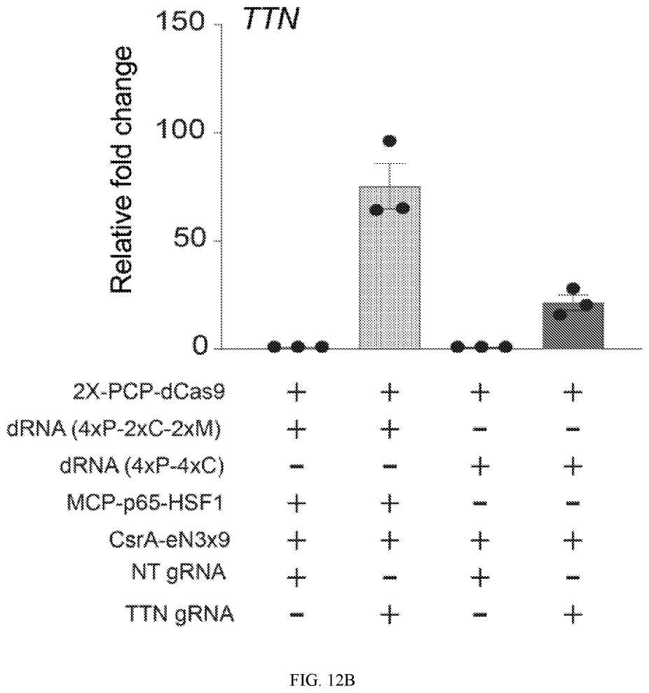

Synergy is a powerful process wherein the effect of recruiting multiple effectors results in a higher than additive modulation compared with each individual component [PMID: 25494202 PMID: 35271371 PMID: 33838111]. Multipartite effector fusions show the potential of assembling multiple endogenous proteins and/or viral based TADs [PMID: 25730490]. However, both cell type and genomic targeting play a large role in relative effector potency. Therefore, identifying endogenous protein synergy in a cell type of interest is a worthwhile goal. The modular nature of dRNA allow user defined control over SL identity and number. Delivering the 4×PP7-2×MS2-2×RAT dRNA along with 2×PCP-dCas9, and gRNA along with different combinations of MCP fused to effector 1 and LicT fused to effector 2 identified synergy between VP64 and MSN or TET1 which is known to initiate the DNA demethylation process (FIGS. 11A-11C). The DRIMER system also identifies previously discovered synergy between the mechanosensitive transcription factor-based TADS MRTFa, STAT1, and eNRF2 previously engineered in the inventors' lab, as they have demonstrated 3 different effector protein recruitment using 3 different SL:SLBP (MCP, CsrA, LicT) pair along with PP7:PCP used for dCas9 targeting gene regulatory element. (FIGS. 11D and 11E). Additional experiments were conducted to minimize delivery constructs required for combinatorial recruitment to prove synergy between the eN3×9 and p65-HSF1 effectors and with single gRNA targeting of TTN utilizing transient transfection (FIG. 12).

Here, the inventors have utilized numerous orthogonal bacterial and viral stem loop (SL) and stem loop binding protein (SLBP) pairs to build a synthetic designer RNA (dRNA) effector recruitment strategy. dRNA is an effective strategy to recruit effectors (transcriptional activators and epigenetic modulators) to dCas9 via SLBP interactions without explicitly modifying gRNA structure. The novel DRIMER platform not only relies on structured intermediate dRNA consists of numerous user-defined SLs, it is also amenable to modification such as incorporation of a riboswitch to build the RiboDRIMER platform, which could be used to precisely tune transcription in an external chemical stimulus dependent manner. Further, using the Opto-DRIMER platform, optical control over transcriptional activation was achieved. Incorporation of the RAT SL coupled with the light controlled protein (VVD) fused to the LicTCAT SLBP domain with a transcriptional effector facilitates transcriptional modulation in a light dependent manner. Overall, multi-modal applications of dCas9-based transcriptional modulation, spatiotemporal regulation of transcriptional output, precise tunability of gene expression completely relies on a programmable intermediate, facultative, functional, designer RNA which are amenable to diverse array of modifications according to specific need for controlling biological phenomenon. Of the best of this platform, the system does not require extra modification in gRNA, transcriptional effector module and dCas9.

Further, to increase the orthogonal SL:SLBP binding pairs in our toolbox, the inventors proved that the bacterial CsrA protein and CsrB RNA which has been shown to bind approximately 18 CsrA proteins could be leveraged to potently activate gene transcription [PMID: 16923806 PMID: 11567002]. With further characterization and engineering, short CBΔ SLs were incorporated into structured dRNA and shown to potently induce transcription through CsrA mediated effector recruitment. Indeed, the CsrA SLBP shows similar potency when recruiting effectors as compared to the widely used MCP SLBP while being more compact (47% smaller). Subsequent experiments demonstrated that the CBΔ:CsrA binding pair could potently recruit effector proteins alone and in combination with the previously described (MS2:MCP, PP7:PCP and RAT:LicT) binding pairs.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
KR102004076B1
US20180057810A1
US20190249230A1
Chao et al., (2008). Structural basis for the coevolution of a viral RNA-protein complex, Nat Struct Mol Biol, 15:103-105, PMID: 18066080.
Chavez et al., (2015). Highly efficient Cas9-mediated transcriptional programming, Nat Methods, 12:326-328, PMID: 25730490.
Cunningham-Bryant et al., (2019). CRISPR-Cas-Mediated Chemical Control of Transcriptional Dynamics in Yeast, Chembiochem, 20:1519-1523, PMID: 30710419.
Dominguez et al., (2022). CRISPR-Mediated Synergistic Epigenetic and Transcriptional Control, CRISPR J, 5:264-275, PMID: 35271371.
Dubey et al., (2003). CsrA regulates translation of the Escherichia coli carbon starvation gene, cstA, by blocking ribosome access to the cstA transcript. J Bacteriol, 185:4450-4460, PMID: 12867454.
Dumont et al., (2016). Human cell lines for biopharmaceutical manufacturing: history, status, and future perspectives, Crit Rev Biotechnol, 36:1110-1122, PMID: 26383226.
Gudapaty et al., (2001). Regulatory interactions of Csr components: the RNA binding protein CsrA activates csrB transcription in Escherichia coli. J Bacteriol, 183:6017-6027, PMID: 11953318.
He et al., (2022). Programmable Transcriptional Modulation with a Structured RNA-Mediated CRISPR-dCas9 Complex. Journal of the American Chemical Society, 144 (28), 12690-12697.
He et al., (2022). Programmable Transcriptional Modulation with a Structured RNA-Mediated CRISPR-dCas9 Complex, J Am Chem Soc, 144:12690-12697, PMID: 35792375.
Kent & Dixon, (2020). Contemporary Tools for Regulating Gene Expression in Bacteria, Trends Biotechnol, 38:316-333, PMID: 31679824.
Konermann et al., (2015). Genome-scale transcriptional activation by an engineered CRISPR-Cas9 complex, Nature, 517:583-588, PMID: 25494202.
Lainšček et al., (2018). Delivery of an Artificial Transcription Regulator dCas9-VPR by Extracellular Vesicles for Therapeutic Gene Activation, ACS Synth Biol, 7:2715-2725, PMID: 30513193.
Liu et al., (2022). Optogenetic control of RNA function and metabolism using engineered light-switchable RNA-binding proteins, Nat Biotechnol, 40:779-786, PMID: 34980910.
Luo et al., (2021). Hepatic stellate cell reprogramming via exosome-mediated CRISPR/dCas9-VP64 delivery, Drug Deliv, 28:10-18, PMID: 33336604.
Ma et al., (2016). Multiplexed labeling of genomic loci with dCas9 and engineered sgRNAs using CRISPRainbow, Nat Biotechnol, 34:528-530, PMID: 27088723.
Ma et al., (2018). CRISPR-Sirius: RNA scaffolds for signal amplification in genome imaging. Nature methods, 15 (11), 928-931.
Mali et al., (2013). CAS9 transcriptional activators for target specificity screening and paired nickases for cooperative genome engineering, 31:833-838, PMID: 23907171.
Martella et al., (2019). Systematic evaluation of CRISPRa and CRISPRi modalities enables development of a multiplexed, orthogonal gene activation and repression system. ACS Synthetic Biology, 8 (9), 1998-2006.
Mercante et al., (2006). Comprehensive alanine-scanning mutagenesis of Escherichia coli CsrA defines two subdomains of critical functional importance, J Biol Chem, 281:1832-1842, PMID: 16923806.
Mercante et al., (2009). Molecular geometry of CsrA (RsmA) binding to RNA and its implications for regulated expression, 392:511-528, PMID: 19619561.
Morita et al., (2020). Synergistic Upregulation of Target Genes by TET1 and VP64 in the dCas9-SunTag Platform, Int J Mol Sci, 21:1574, PMID: 32106616.
Nguyen et al., (2021). CRISPR activation of long non-coding RNA DANCR promotes bone regeneration, Biomaterials, 275:120965, PMID: 34147719.
Nishimasu et al., (2015). Crystal Structure of Staphylococcus aureus Cas9, Cell, 162:1113-1126, PMID: 26317473.
Nowak et al., (2016). Guide RNA engineering for versatile Cas9 functionality, Nucleic Acids Res, 44:9555-9564, PMID: 27733506.
Nuñez et al., (2021). Genome-wide programmable transcriptional memory by CRISPR-based epigenome editing, Cell, 184:2503-2519, PMID: 33838111.
Peabody, (1993). The RNA binding site of bacteriophage MS2 coat protein, EMBO J, 12:595-600, PMID: 8440248.
Reichenbach et al., (2023). A lentiviral vector for the production of T cells with an inducible transgene and a constitutively expressed tumour-targeting receptor, Nat Biomed Eng, 7:1063-1080, PMID: 37069267.
Rissone et al., (2022). Stem-loop formation drives RNA folding in mechanical unzipping experiments, Proc Natl Acad Sci USA, 119: e2025575119, PMID: 35022230.
Ritchie et al., (2007). RNA stem-loops: to be or not to be cleaved by RNAse III, RNA, 13:457-462, PMID: 17299129.
Schoger et al., (2020). CRISPR-Mediated Activation of Endogenous Gene Expression in the Postnatal Heart, Circ Res, 126:6-24, PMID: 31730408.
Siew et al., (2021). Downstream processing of recombinant human insulin and its analogues production from E. coli inclusion bodies, Bioresour Bioprocess, 8:65, PMID: 34336550.
Taghbalout et al., (2019). Enhanced CRISPR-based DNA demethylation by Casilio-ME-mediated RNA-guided coupling of methylcytosine oxidation and DNA repair pathways, Nat Commun, 10:4296, PMID: 31541098.
Weber & Fussenegger, (2007). Inducible product gene expression technology tailored to bioprocess engineering, Curr Opin Biotechnol, 18:399-410, PMID: 17933507.
Zalatan et al., (2015). Engineering complex synthetic transcriptional programs with CRISPR RNA scaffolds. Cell, 160 (1-2), 339-350.
Zalatan et al., (2015). Engineering complex synthetic transcriptional programs with CRISPR RNA scaffolds, Cell, 160:339-350, PMID: 25533786.
Zhao et al., (2021). Investigating crosstalk between H3K27 acetylation and H3K4 trimethylation in CRISPR/dCas-based epigenome editing and gene activation, Sci Rep, 11:15912, PMID: 34354157.

1. A composition comprising one or more nucleic acid(s) encoding (i) a first effector protein fused to a first stem-loop binding protein, wherein the first effector protein is a CRISPR/Cas protein, (ii) a second effector protein fused with a second stem-loop binding protein, (iii) an RNA molecule comprising at least one of a first stem loop that binds to the first stem-loop binding protein and at least one of a second stem loop that binds to the second stem-loop binding protein, and (iv) a guide RNA(s) (gRNA) targeting at least one genomic locus.

2. The composition of claim 1, wherein the stem loop:stem-loop binding protein pairs comprise PP7:PCP, MS2:MCP, CsrB-SL:CsrA, RAT:LicT, and/or RAT:LicV.

3. The composition of claim 1, wherein the CRISPR/Cas is SadCas9, CjdCas9, SpdCas9, SpRY dCas9, or HIFI dCas9.

4. The composition of claim 1, wherein the first effector protein is fused to two or more copies of the first stem-loop binding protein.

5. The composition of claim 1, wherein the gRNA and the RNA molecule are each under the control of a polIII promoter.

6. The composition of claim 1, wherein the RNA molecule comprises two, three, four or more of the first stem loop; and two, three, four or more of the second stem loop.

7. The composition of claim 1, further comprising a third effector protein fused with a third stem-loop binding protein, and wherein the RNA molecule further comprises at least one of a third stem loop that binds to the third stem-loop binding protein.

8. The composition of claim 1, wherein the RNA molecule comprises 2-4 PP7 stem loops, 2-4 MS2 stem loop, and 2-4 RAT stem loops, and wherein one effector protein is fused to PCP, one effector protein is fused to MCP, and one effector protein is fused to LicT or LicV.

9. The composition of claim 8, wherein the RNA molecule further comprises at least two CsrB-SL stem loops, wherein one effector protein is fused to CsrA.

10. The composition of claim 1, wherein each of the effector proteins is, independently, a transactivating domain or epigenetic editor.

11. The composition of claim 1, wherein each of the effector proteins is, independently, selected from VP64, eN3×9, TET1, and MSN.

12. The composition of claim 1, wherein the RNA molecule further comprises a self-stabilized and small molecule/biomolecule induced cleavable OFF-Riboswitch.

13. The composition of claim 1, wherein the RNA molecule further comprises a small molecule/biomolecule stabilized ON-Riboswitch.

14. The composition of claim 1, wherein the interaction between the second stem loop and the second stem-loop binding protein is light dependent.

15. The composition of claim 1, wherein the components are all present on a single nucleic acid molecule.

16. A cell comprising the composition of claim 1.

17. A viral vector comprising nucleic acid sequences encoding (ii) a second effector protein fused with a second stem-loop binding protein, (iii) an RNA molecule comprising at least one of a first stem loop that binds to the first stem-loop binding protein and at least one of a second stem loop that binds to the second stem-loop binding protein, and (iv) a guide RNA(s) (gRNA) targeting at least one genomic locus.

18. A method of modifying a phenotype of a cell comprising introducing the composition of claim 1 into the cell.

19. The method of claim 18, wherein the phenotype is an expression level of one or more genes and/or an epigenetic landscape.

20. An engineered stem loop that specifically binds to CsrA, wherein the engineered stem loop is CsrB-SL Type 1 or CsrB-SL Type 2.


SEQUENCE LISTING (13 sequences)

SEQ ID NO: 1 - RNA, 25 nt, Emesvirus zinderi
  source 1..25: mol_type = transcribed RNA
gcgcacatgaggatcacccatgtgc

SEQ ID NO: 2 - protein, 116 aa, Emesvirus zinderi
MASNFTQFVLVDNGGTGDVTVAPSNFANGIAEISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 3 - RNA, 26 nt, Pepevirus rubrum
  source 1..26: mol_type = transcribed RNA
ataaggagtttatatggaaaccctta

SEQ ID NO: 4 - protein, 127 aa, Pepevirus rubrum
MSKTIVLSVGEATRTLTEIQSTADRQIFEEKVGPLVGRLRLTASLRQNGAKTAYRVNLKLDQADWDCSTSVCGELPKVRYTQVWSHDVTIVANSTEASRKSLYDLTKSLVATSQVEDLVVNLVPLGR

SEQ ID NO: 5 - RNA, 19 nt, Muvirus SfMu
  source 1..19: mol_type = transcribed RNA
ctgaatgcctgcgagcatc

SEQ ID NO: 6 - protein, 62 aa, Muvirus SfMu
MKSIRCKNCNKLLFKADSFDHIEIRCPRCKRHIIMLNACEHPTEKHCGKREKITHSDETVRY

SEQ ID NO: 7 - RNA, 25 nt, synthetic construct
  source 1..25: mol_type = other RNA
attgttactgctacggcaggcaaaa

SEQ ID NO: 8 - protein, 55 aa, Bacillus subtilis
KIAKVINNNVISVVNEQGKELVVMGRGLAFQKKSGDDVDEARIEKVFTLDNKDVS

SEQ ID NO: 9 - RNA, 16 nt, synthetic construct
  source 1..16: mol_type = other RNA
ggcacaaggatgtgcc

SEQ ID NO: 10 - RNA, 14 nt, synthetic construct
  source 1..14: mol_type = other RNA
gcacaaggatgtgc

SEQ ID NO: 11 - protein, 60 aa, Escherichia coli
LILTRRVGETLMIGDEVTVTVLGVKGNQVRIGVNAPKEVSVHREEIYQRIQAEKSQQSSY

SEQ ID NO: 12 - RNA, 359 nt, Escherichia coli
  source 1..359: mol_type = transcribed RNA
gagtcagacaacgaagtgaacatcaggatgatgacacttctgcaggacacaccaggatggtgtttcagggaaaggcttctggatgaagcgaagaggatgacgcagacgcgttaaaggacacctccaggatggagaatgagaaccggtcaggatgattcggtgggtcaggaaggccagggacacttcaggatgaagtatcacatcggggtggtgtgagcaggaagcaatagttcaggatgaacgattggccgcaaggccagaggaaaagttgtcaaggatgagcagggagcaacaaaagtagctggaatgctgcgaaacgaaccgggagcgctgtgaatacagtgctccctttttttatt

SEQ ID NO: 13 - RNA, 270 nt, synthetic construct
  source 1..270: mol_type = other RNA
acgagcagaccatatggggtcgctcgtgatccgaggagcagacgatatggcgtcgctccggctgcagcagcagaggatatggcctcgctgcggagtgaccagcagagcatatgggctcgctgggccaccggcacaaggatgtgcccggacggcacaaggatgtgccagagtagcgcacaaggatgtgcggtgtgtggcacaaggatgtgcccacacaaactactcaaatgtccgaaaggtggcaaacactccaaagcagccaaacggatc